(19) United States
(12) Patent Application Publication
(10) Pub. No.: US 2017/0177807 A1
(43) Pub. Date: Jun. 22, 2017

(54) ENHANCED USER INTERFACE FOR A SYSTEM AND METHOD FOR OPTIMIZING SURGICAL TEAM COMPOSITION AND SURGICAL TEAM PROCEDURE RESOURCE MANAGEMENT

(71) Applicant: GAVIN FABIAN, Los Angeles, CA (US)

(72) Inventor: GAVIN FABIAN, Los Angeles, CA (US)

(21) Appl. No.: 15/385,809

(22) Filed: Dec. 20, 2016

Related U.S. Application Data
(63) Continuation of application No. 62/270,453, filed on Dec. 21, 2015.

(51) Int. Cl. G06F 19/00; A61B 90/98; G06Q 10/08

(52) U.S. Cl. CPC G06F 19/327; G06F 19/322; G06Q 10/087; A61B 90/98

(57) ABSTRACT

A system and method for automatically creating a surgical team schedule is herein provided with enhanced user interfaces with optional biometric recognition apparatus. Typical surgical team participants may include nurses, vendor representatives, administrative and technical staff, doctors and other specialists such as anesthesiologists and surgeons. Optimal participation criteria may include individual staff location, workload, vacation schedules, levels of skill, cost, etc. In addition, automatically managing and optimizing the supplies and medicines utilized in surgical procedures may be achieved to insure and monitor quality and cost control. This is achieved by insuring that all the correct supplies and personnel are timely and locally assembled and available at the correct precise surgical site, that the correct surgical procedure has been specified, and that the correct patient is verified as present on site prior to commencement of the procedure. Image and audible sensors may track personnel, medical supplies and pharmaceutical products.

This application claims priority from U.S. Provisional Patent Application No. 62/270,453, filed on Dec. 21, 2015, and U.S. Provisional Patent Application No. 62/270,444, filed on Dec. 21, 2015, the contents of both which are incorporated herein by reference.

### BACKGROUND OF THE INVENTION

Among known workforce management systems used for scheduling and managing personnel are systems designed to interface directly to personnel who are “on call” or called into service as required or desired. Such systems typically include a basic planning capability to enable a manager to forecast future employee requirements to service needs. Some of these systems provide a scheduling capability which allocates employee work hours according to forecasted staffing requirements. Employees are assigned to fill the schedules and employee assignments are posted.

In a hospital setting, for many years, the normal process has included employing a Surgery Scheduler whose responsibility it is to assemble a surgical team for each procedure that has been set, and then, assign the procedure for one of a multitude of “operating rooms”. The team is staffed with particular staff depending upon what is entailed to complete the procedure. For example, for a typical hip replacement, various nurses will be required for the team; a surgeon and an assistant to the surgeon; an anesthesiologist and a vendor representative from the company responsible for manufacturing the hip “ball and socket” assembly. Others on the team may include various surgical staff and administrative personnel as required. In addition, a surgical procedure “parts list” is compiled. It may include various “sharps” for cutting; IV bags filled with medical products or maybe even extra blood; and basically, all the surgical supplies and pharmaceutical products and medicines that need to be available inside the operating room. Last but not least, the patient is paired up with the team, and an operating room is assigned.

Things go wrong. Sometimes various team members do not show up, or sometimes, they are late or end up at the incorrect operating room. Sometimes, the wrong patient is brought to a particular operating room. Sometimes the wrong medicine or medical supplies are provided to an operating room. In order to minimize against these and other problems, a surgical scheduler and coordinator is hired to oversee all of the surgical resources, including personnel and supplies. Widely used, the scheduler will often use a “wet board” in an operating room setting, with lists by operating room, so various staff members will know where they need to be and when, and which patient is in each operating room, so that the correct medical supplies and medicines may be provided to the correct operating rooms. The possibility of human error is significant if not substantial. And the price for inaccuracy may be severe, even resulting in death.

Known workforce management systems do not account for the many factors that can influence workload demands and forecasting. Workload, expertise, and external factors such as vacation schedules, or even level of qualification can become very significant in a hospital setting where human life or quality of life is at stake. On the other side of the spectrum, if healthcare providers and surgery center operators and hospitals simply “err to the side of caution”, always, by over providing and over sourcing personnel and medical supplies and medicines, and surgical supplies no less which may be costly, the result is to cause healthcare expenses to needlessly soar out of control. In the prior art, systems are all or nothing. Manual human monitoring and control is the norm, and it's both inefficient and subject to error. Consequently, if an error occurs and life is lost or a patient is substantially impaired, insurance costs soar, and again, costs become uncontrollable.

Traditional workforce management systems in the prior art fail to address any of the needs of the surgical center or operating room management field. What is needed in the art and has not been available is a scheduling system and method, which dynamically incorporate surgical personnel data along with surgical procedure supply and patient data, optimized and referenced to generate an optimal schedule, and then, monitoring real life implementation of that schedule in order to minimize human error. In parallel, as various surgical procedures are scheduled, a supply and medicine list is built per procedure, so that inventory management is achieved and the correct supplies and medicines are appropriated to the correct procedure and the correct location within a surgery facility. And last but not least, the correct patient with the correct patient medical records are coordinated in parallel so that all three workflows or sets of criteria may be merged and then monitored, almost like the assembly line of an automobile manufacturing.

Managing professional, medical or technical personnel services typically involves multiple processes, departments, systems, and personnel that have suffered from no or poor integration in the past. Conventional systems focus on the primary process of performing physical work (clock in, clock out, etc.) are simply not sufficient where many factors dictate who should be included in a particular operating room and associated surgical procedure, and, which patient gets which team, depending on medical necessity, cost, insurance coverage, usual and prevailing rates and qualities of care, and so on.

To have an effective and efficient system, surgical centers or hospital surgery departments need to define, measure, and track labor resources, material resources, surgical equipment, supplies and medicines, insurance requirements and quality of care requirements and patient expectations, not to mention the financial objectives and incentives of overall healthcare networks, whether for or not for profit. In conventional systems, however, most of these objectives are managed manually (or some are not managed at all), incurring high labor costs, or are partially done with only rudimentary work order automated processes and little to no system integration, which is a problem, both in terms of cost control and quality control (which in this field may amount to life and death).

### SUMMARY OF THE INVENTION

The present invention provides a system and method for optimizing a surgical team composition and optimizing and monitoring or managing a surgical team's resources, and includes advanced audio and video sensors that may serve to enhance the user interface of the present invention.

Traditionally, surgery centers or surgery departments within hospitals employ schedulers to organize surgical procedures. As an initial matter, a surgical procedure may be prescribed by a doctor, to be performed on a particular patient. Depending upon the condition of the patient and the complexity of the procedure, the scheduler must assemble a team of personnel to perform the procedure and must assemble a list of what will be needed in terms of materials and medicines to successfully complete the procedure. In addition, the condition of the patient must be taken into account. For example, if the patient has an allergy to latex, no latex may be used. If the patient has a heart condition, the anesthesiologist, for example, may need to vary his or her procedures. Furthermore, for example, a patient with an infectious may need to have a certain team assembled and a certain quarantined operating room may need to be assigned. It is a principal object of the present invention to provide a method and system for automating the foregoing process by a series of data input mechanisms, monitoring mechanisms, data base utilization and cross-referencing, data processing and a user interface (UI) that is simple for surgical facility personnel, patients and relevant constituencies to utilize to stay informed, be held accountable and directed as appropriate to maintain desired levels of quality control, safety and cost control.

Data input mechanisms may be as simple as keyboards, smart phones, audio transducers or RFID devices. Data input may also be obtained and provided by video cameras, laser sensors, wearable devices like medical probes or even smart watches, or really any way to collect data from all of the users of the system. Monitoring mechanisms may be used to sense the precise sequence of events for each procedure, and databases accessed and cross-referenced so that all of the processes are automatic.

Enhanced user interfaces are an important aspect of the present invention. The present invention may best be implemented in part by way of a web site and smart phones, which may access a web site or even work via a software application or “app”. Smart phones possess many ways to alert and sense user input including a microphone for audible signals, a shock sensor, altimeter functionality, global positioning system (GPS) capability, and a camera for image detection or video processing. Furthermore, smart phones have touch screens for logging tactile response from humans, and for processing signals for other electronic devices proximate to humans, including connectivity with wearable technology such as watches or other human sensors that monitor the movement and behavior of users. In addition, the present invention may provide that video sensors or cameras are distributed in any location important to surgical procedures, along with microphones to sense audible or noise signals. Audio and video transducers may be distributed in hospitals or surgical centers, such as in waiting rooms, patient intake areas, pre and post operation areas, operating rooms, and even storage rooms where medical supplies and pharmaceutical products are stored. In this manner, the exact status and position of all surgical procedure resources may be monitored to insure that they are in a proper position to be integrated into a surgical procedure workflow at particular times in order to optimize the monitoring and processing of surgical procedures. In this matter, quality control and safety may be monitored and achieved in an optimal sense, while costs monitored and controlled from local or remote locations.

Remote monitoring of surgical procedures and remotely assembling human and material resources for a surgical procedure is best accomplished by use of the present invention. By tracking each and every element and resource required for a surgical procedure, and knowing in advance that an element or resource is “out of place” or unavailable, administrators will know in advance of changes that need to be made in order to maintain schedules and provide patients with required levels of care. For example, if a snowstorm hits an area and a particular anesthesiologist is going to be late for a procedure, the system according to the present invention will notify administrators the human resource is “out of spec” and can then alert administrators to either replace the missing resource or delay the procedure. In that way, quality and cost standards are enhanced. Safety is enhanced, and third party payers such as insurance companies are not taxed for avoidable cost excesses.

Monitoring humans involves making sure that their movements are tracked, including within an operating room. For example, by use of the present invention, it is possible to recognize who is within an operating room so that no procedure progresses until all the required parties are present. Likewise, the present invention may sense that the correct patient is in the correct operating room with the wholly correct surgical team. RFI© bracelets and image scanning (via a camera for example) may be used to insure that the correct people are in the correct places at the correct time.

Furthermore, according to the present invention, microphones may be used to record or sense audio signals. In this manner, anything important, such as emergency conditions, may be immediately sensed by facility administrators.

This may be accomplished via stationary microphones placed within designated areas or via microphones contained within cell phones. In addition, video display panels and loud speakers may be integrated into the present invention, either via fixed means or via hand held smart phones or tablets. In this manner, audio interfaces like the Apple Siri interface may be utilized to guide surgical procedure human resources through all facets of the process, from waking up in the morning, reporting to work, scrubbing up for a procedure, guiding them through the procedure (perhaps privately via a Bluetooth earpiece), and then throughout the rest of the post op process, only to repeat the sequence until they are off duty.

In all cases, GPS and other proximity sensors can be managed and taken into account, along with all other environmental factors such as weather, and so forth.

By way of example according to the present invention, let's suppose a surgical procedure is prescribed by a doctor, and the patient, doctor and the patient's insurance company have agreed on the use of a particular surgical center to perform the procedure. In that case according to the present invention, once assigned, the present invention will replace many if not all of the functions of the traditional surgical procedure scheduler. According to the present invention, the request for a surgical procedure is assigned to a given surgical procedure center, whether that center be for profit or not for profit or stand-alone or in a hospital. In all cases, the surgical center has an administrator who may utilize the present invention to maximize quality control, safety and cost control.

In accordance with one embodiment of the present invention, a method for centrally creating a schedule is described for use in connection with a distributed network, which includes a host server and at least one first client side machine. Various hospitals and surgical centers across the country may tie into one central control center, without business and medical being shared between clients, and all HIPAA compliant. According to the present invention, schedule requirements provided by the first client side machine through the distributed network are processed, for example, at the host server. A schedule is then constructed in accordance with the processed schedule requirements. A plurality of data sources may provide further information to the host server through the distributed network.

According to an enhanced version of the present invention, doctors and nurses may be crowd sourced so that various surgical procedures may be “bid out” so that medical professionals are assembled based on availability and pricing. In that way, a market for surgical procedure specialists or even anesthesiologists can be established by geographic market, with various hospitals and surgery centers drawing from a collection of local pooled talent. Accordingly, the schedule may be revised in accordance with any further information that is received, and the revised schedule is made available to each of the first client side machines that are connected in the distributed network.

In further aspects of this first embodiment, an optimal shift pattern or optimal staffing requirement can be determined for the schedule. In a particularly preferred embodiment, the host server communicates with one or more second client side machines, which can provide shift requests to the host server.

So according to the present invention, the surgery control system starts by assigning for a particular patient the personnel preferred for a particular procedure and ascertaining what supplies and medicines will be required for the procedure. The patient has an associated data file with a complete medical history and list of patient information such as name, contact information, location, age, gender, allergies, other medical conditions, family relationships and of course billing and insurance information. In short, a patient presents itself to the surgical team with all its conditions and illnesses and the surgical team gets paid to perform the desired procedure in a safe, medically effective and cost effective manner.

The surgery personnel selection process is complex. For each surgical procedure, medical personnel are provided, including nurses, doctors, surgeons, anesthesiologists and other administrative personnel associated with the surgical facility. In addition, various equipment or vendor representatives may need to be included. For example, in certain orthopedic procedures such as a hip replacement, the manufacturer of the ball joint used for the hip replacement may want to send a representative to the surgery to insure that it is installed properly. In all cases, the available future dates and locations of each person need to be taken into account. The skill levels and necessity of each person needs to be taken into account. The cost associated with each person needs to be taken into account. In all cases, each participant proposed for a surgical procedure must be compared against a list of what is required in terms of safety, effectiveness and cost, and then, an optimization process is conducted to automatically crowd source the appropriate staff for a given procedure at a given time and place. Likewise, the time and place may be optimized based on availability of suitable operating rooms given the patient needs and desires. By performing the staff set formation process automatically, workloads may be balanced so that individual medical staff members do not become fatigued, which in turn may increase safety, increase safety and minimize negative outcomes.

According to the present invention, once staff, time and place are optimized and set for a particular procedure for a particular patient, all the criteria for each participant becomes a part of the process status file. At that point, required medical supplies, medical devices, medicines and other special needs are sourced and earmarked within inventory or placed into an order cue. At any point, the unavailability of any resource, whether it be personnel or supplies or medicines, may cause a change in the date or place or even substitution of personnel, supplies or medicines. Furthermore, at this point, a cost file is created of anticipated costs for all surgery resources, so that insurance companies and patients may input data to approve certain charges while restricting others, thus influencing the level of care provided.

As the surgical procedure date approaches, the system according to the present intervention interconnects all constituencies. All medical staff members are kept aware of the upcoming procedure via calendar, emails and other communication mechanisms. For example, the user interface may be an “app” running on a smart phone, so that all medical personnel and patients and their constituencies may know the status of the upcoming procedure and whether any changes in time, date, place, supplies, medicines or staff have occurred. Likewise, surgical administrators and third party payers or insurance companies may be “in the loop”. At any point, if a particular procedure is not being handled according to that which is desired by all relevant constituencies, a decision may be made to change the composition of the procedure (staff, supplies, medicines or site), postpone it or even cancel it. For example, the death of a patient by any means would result in the automatic cancellation of a planned surgical procedure, naturally.

Once the surgery date and time have arrived, various data input means are engaged. Various data access levels are established so that administrators and doctors have a certain level of access, while other staff and patients may have a different level yet, and so forth. Access credentials can influence both what a given person may enter into the system and what information they may be able to retrieve. In all cases, data security is a priority due to the sensitive nature of medical information. While keyboards and automatic audio transcription devices and systems may be used to cue up events and record information according to the present invention, it is anticipated that smart cameras will begin to play a larger role in the future. For example, if a total of five (5) particular medical staff members are assigned to a particular operating room, a smart camera may be able to recognize who is in there and then, the system according to the present invention may inform the staff who have entered the incorrect operating room.

Automatic data input via wearable technology in the future can enhance the performance of the present invention. Everything from RAD bracelets on patients to medical staff smart phones and smart watches to garments with telemetry sensors and electronic eyeglasses that utilize projected images. Data collection and data status with a feedback loop insure that the surgery “game plan” and execution is optimized from surgical procedure inception through to the patient recovery room. By use of the present invention, expensive staffing levels may be managed, expensive inventory procurement procedures may be streamlined and overall efficiency achieved, while insuring that only the correct processes are being undertaken. It is contemplated that medical staff actions such as surgeon movements may be monitored and compared with that particular surgeon's own norms, so that out of the norm conditions are spotted and staff are given a chance to change a course of direction to achieve enhanced levels of safety and efficiency. During the actual surgical procedure, the use of supplies and medicines may also b e tabulated and compared to norms. According to the present invention, said norms may be the norms established for that surgical center based on its history or by its administrator, or the norms may be established regionally, nationally and internationally. Accordingly, if a particular surgical procedure entails a certain number of movements by each of the participants in an operating room, and at any point during a procedure the norms for levels of activity monitored appear to be “out of spec”, the surgical team may be warned either visually or by audio transducer.

A primary aspect of the present invention is to establish a game plan and then monitor the execution of that game plan, so as to increase safety, reduce cost, and increase efficiency. Another primary feature of the present invention is to utilize common user interfaces such as smart phones to communicate with medical staff members, patients and all related constituencies. By one variation of the present invention, the user interfaces by be in the form of software “apps” or applications, and available for distribution via various “app” stores.

These and other features, embodiments, and aspects the present invention can be appreciated from the following drawing description and detailed description of a preferred embodiment.

Other features and aspects of the disclosed technology will become apparent from the following detailed description, taken in conjunction with the accompanying drawings, which illustrate, by way of example, the features in accordance with embodiments of the disclosed technology. The summary is not intended to limit the scope of any inventions described herein, which are defined solely by the claims attached hereto.

### BRIEF DESCRIPTION OF THE DRAWINGS

FIG. 1 is an overall view of the present invention.

FIG. 2 is a block diagram, which describes services offered through the mobile application.

FIG. 3 is a block diagram, which describes the procedure database in the administrator interface.

FIG. 4 is a block diagram, which describes the employee workflow interface.

FIG. 5 is a block diagram, which describes the administrator scheduling interface and outgoing workflow interface.

FIG. 6 is a block diagram, which describes the procedure workflow interface.

FIG. 7 is a block diagram, which describes client administrator data aggregation.

FIG. 8 is a block diagram, which describes the communication interface.

FIG. 9 is a block diagram, which describes the patient profile interface.

FIG. 10 is a block diagram, which describes the client administrator employee profile interface.

FIG. 11 is a block diagram, which describes the client administrator geo-location monitoring system.

FIG. 12 is a block diagram, which describes employee identity verification interface.

FIG. 13 is a block diagram, which describes inbound data integration between the client's internal server and the online application server.

FIG. 14 is a block diagram, which describes the outbound data flow within the client interface.

FIG. 15 is a block diagram, which describes the case proposal functionality for the client system.

FIG. 16 is a block diagram, which describes the mobile application features available to the client for accessing case data files.

FIG. 17 is a block diagram, which describes the aggregation of data that is displayed within the user interface.

FIG. 18 is a block diagram, which describes the user interface when accessed by a system user.

FIG. 19 is a block diagram, which describes a possible overall user interface according to the present invention.

FIG. 20 is a block diagram, which describes a display board interface, which may be used by an administrator user according to the present invention.

FIG. 21 depicts an example of an embodiment according to the present invention, which describes the login parameters for the application interface.

FIG. 22 depicts an example of an embodiment according to the present invention, which describes the case list data overview of the application interface.

FIG. 23 depicts an example of an embodiment according to the present invention, which describes the filter and sort functionalities of the application interface.

FIG. 24 depicts an example of an embodiment according to the present invention, which describes the filter and sort functionalities of the application interface.

FIG. 25 depicts an example of an embodiment according to the present invention, which describes the filter and sort functionalities of the application interface.

FIG. 26 depicts an example of an embodiment according to the present invention, which describes the display mode of the application interface.

FIG. 27 depicts an example of an embodiment according to the present invention, which describes the details functionality of the application interface.

FIG. 28 depicts an example of an embodiment according to the present invention, which describes the requests function of the application interface.

FIG. 29 depicts an example of an embodiment according to the present invention, which describes the case team and documents overview of the application interface.

FIG. 30 depicts an example of an embodiment according to the present invention, which describes the activity overview of the application interface.

FIG. 31 depicts an example of an embodiment according to the present invention, which describes the notification functionality of the application interface.

FIG. 32 depicts an example of an embodiment according to the present invention, which describes the settings functionality of the application interface.

FIG. 33 depicts an example of an embodiment according to the present invention, which describes the settings functionality of the application interface.

FIG. 34 depicts an example of an embodiment according to the present invention, which describes the settings functionality of the application interface.

FIG. 35 depicts an example of an embodiment according to the present invention, which describes the propose a case module.

FIG. 36 depicts an example of an embodiment according to the present invention, which describes the various user groups of the application.

FIG. 37 depicts an example of an embodiment according to the present invention, which describes the various platforms available to the user to access the application.

FIG. 38 depicts an example of an embodiment according to the present invention, which describes the use of the application on a mobile device.

FIG. 39 depicts an example of an embodiment according to the present invention, which describes the digital surgery board feature of the present invention.

FIG. 40 depicts an example of an embodiment according to the present invention, which describes the digital surgery board feature of the present invention.

FIG. 41 depicts an example of an embodiment according to the present invention, which describes the digital surgery board feature of the present invention.

FIG. 42 depicts an example of an embodiment according to the present invention, which describes the digital surgery board feature of the present invention.

FIG. 43 depicts an example of an embodiment according to the present invention, which describes the digital surgery board feature of the present invention.

### DETAILED DESCRIPTION OF THE PREFERRED EMBODIMENT

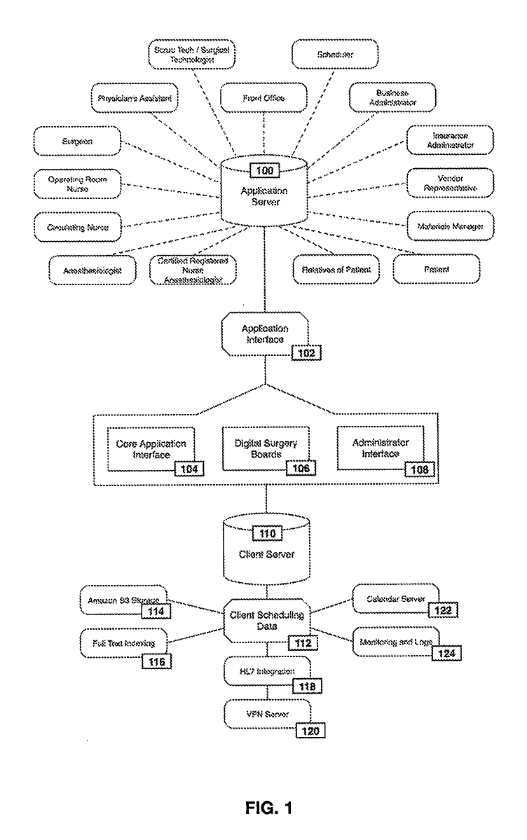

FIG. 1 is an overall view of the present invention. According to the present invention, a cloud-based application server (100) may be used to enable all components and subsystems to interact with one another without loss of communication. Of course, local servers may be employed to insure continuity during communications outages or power outages. By employing a distributed data server approach, the overall system shown in FIG. 1 may be implemented and deployed using servers located at any number of local or geographically diverse locations. It is envisioned that an operator and user of the present invention will locate its top-level administrative headquarters at one or a few off-site geographic locations. That is, off-site from the various hospitals or surgery centers. It is a principal object of the present invention to keep track of all resources deployed within hospitals and surgical centers throughout a health care chain, or even multiple healthcare chains or facilities, throughout the world. Consequently, it is a principal object of the present invention to keep track of all resources deployed within hospitals and surgical centers throughout a healthcare chain or even multiple healthcare facilities throughout the world. Client servers (110) may be placed at any single location or multiple locations with backups at off-site locations. According to the present invention a local physical server may connect to a cloud server so various input and output devices such as digital surgery boards may be located throughout any healthcare facility or surgical center.

According to the present invention, a digital surgery board (106) may be deployed so that various users of a hospital or surgery center (medical professionals, administrators, patients, equipment vendors, medical product or pharmaceutical representatives, patients, patient families and other users) may know the status of a patient, both from a patient advocate or family perspective and from a need to know from a hospital or surgical staff or staff affiliate perspective. A digital surgery board (106) may serve as an interactive kiosk with more functionality then a display board without input means so that some boards accept certain user input while other boards, such as boards in a waiting room, are output equipped and configured only. All surgery boards may be browser-based and may use display mechanisms, which are very familiar to the public, akin to for example status boards contained within airports to indicate flight status. All surgery boards may be wireless so that they can work from a wireless network, a public telephone network or compatible public wireless network. Consequently, every surgery board (106) becomes or may become an input device or an output device.

By way of example, suppose a hospital waiting room is equipped with a patient status board, and provides output only. Then, suppose a hospital administrator wanted to add a feature whereby a family member may touch a particular patient listed, input a code only known by family members, and in turn, page a staff member to get a verbal update, face to face. With the present invention, said functionality may be deployed anywhere throughout the world. By way of example, with the present invention, family members and surgeons and all the medical procedure constituencies in between may receive and enter information to monitor, track and provide data pertaining to a surgical procedure, somewhat in the same way a consumer may place, monitor, track and even modify an order placed via the internet scheduled for home delivery.

Surgery boards may be located anywhere within a hospital such as the front desk or first patient intake or admission area on a pc, a pre-op group, an operating room, a post-op or so-called “PACU (post anesthesia care unit)”, a waiting room or even a visitor's lounge. And, each display may be configured to display only selected data and may be configured to accept data or certain data only, or no data at all. RF beacons may be associated with various physical displays so that users of portable electronic devices are able to “sync up” so that a local surgery reporting board and a portable information display are complementary.

In a cloud structure, the present invention may be implemented via the use of well-known cloud server platforms that are HIPAA compliant with HL7 (118) and VPN (120) capability. A calendar database (122) is important as procedures need to be scheduled well in advance, and care must be taken to insure that the correct and ample resources are available for each procedure, while not over providing for any procedure, in order to efficiently operate an overall facility.

A major advantage of the present invention is to make sure that the correct personnel and the correct medical supplies and drugs are at the right place at the right time, ready for attention to the right patient in the right place at the right time. Some medical supplies and pharmaceutical products have long lead ordering times, so therefore, need to be ordered well in advance of the day of the surgical procedure. Calendars (122) and timers are a principal feature of the present invention so that surgical procedures are scheduled and the correct resources (personnel, drugs and supplies) are on site in a timely manner. All of this “schedule building” (112) must take place on a server that is internally HIPAA compliant as sensitive medical data is at stake. Monitoring logs (124) and data base programming may be accomplished by any number of systems. A software application according to the present invention may incorporate deployment reports, transactional tracing, cross application tracing and alert policies. All of this insures to the best possible means that everyone and everything is where it needs to be before, during and after a surgical procedure. Furthermore, that quantities and pricing are traced along with patient medical information.

According to the present invention, data is gathered and logged to enhance safety within a medical facility in order to save lives and promote better health.

Data may also be sorted and analyzed in order to promote efficiency and to insure that medical insurance realities associated with each patient are in compliance to minimize the possibility that a particular patient will be presented with unexpected medical expenses. Data logging according to the present invention, therefore, needs to be sufficiently detailed. Accordingly, log management and analytics program that collects and analyzes data found within log files (124), The data is collected and analyzed in real-time across log file software stacks. The present invention may use a pre-processed layer in order to filter, correlate and visualize the data log. The analyzed data can be easily accessed through a search engine to determine the amount of specific events that have been logged in a specified time period. Other search capabilities may include average and sum analysis on key value pairs within the log data that can be generated as visualizations of key metrics. The collected data and subsequent analytics may then be stored on a cloud based platform server. All client program data from incoming log files is backed up to an onsite or offsite and-or cloud server (114). The collected and analyzed data is then easily accessible to read and develop, implementing an open application program interface, or API, The present invention can oversee all data logging related operations related to client performance management. Furthermore, the present invention analyzes each segment of collected data through a development operations perspective in order to determine the percentage of data that is useful and actionable and what percentage of data is redundant. This analysis ensures that the client program is running at optimal efficiency. With the advent of artificial intelligence, the present invention may simulate one or more thinking people and uses text and/or speech to interact with users. The present invention provides user simplicity within the high paced medical procedure environment, and can be readily adapted to take advantage of the major advancements of artificial intelligence systems. For example, the present invention may extract concepts from text and/or speech and utilize numeric representations of concepts and their relationships. The extraction of concepts would allow the expression in various patterns to be understood by the system. The processes could allow the system to bypass human language constraints in order to think in concepts. The present system may also dynamically construct output so that it can generate intelligent responses in a number of grammatically correct ways. In all cases, insuring patient safety is of paramount importance.

In addition, the ability to search through substantial volumes of data is an important feature according to the present invention. One search feature of the present invention provides a search capability within a client program by using an externally hosted search engine. In that case, the program may index only the client data to provide streamlined and specified search capability. This search model is intended to give performance advantages of a full in-house search engine operating on the client program back-end database, also implementing a simplified and site restricted search engine. By using advanced search engine techniques, the present invention may provide a rapid response from searching specified client data rather than an entire web infrastructure (116). Searchable data can be customized to client specifications, data structure, and metadata facets. As a result, the relevance of search results is improved as searching can take semantics of client data content into account. Such a capability is particularly important in the medical environment, where semantics are especially prevalent.

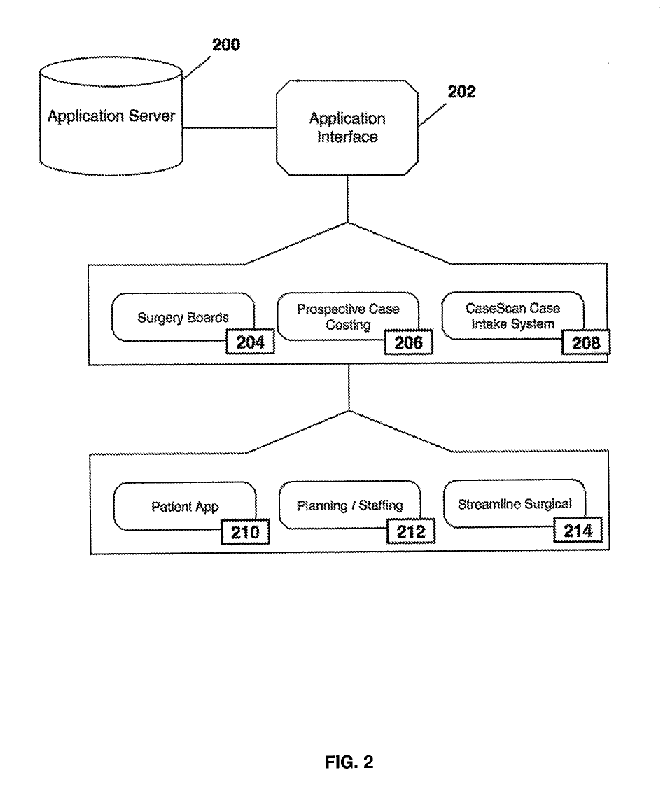

FIG. 2 is a block diagram, which describes services offered through the software application interface according to the present invention. In accordance with the preferred embodiment of the present invention, the application program interface (202) or API displays data stored in the application server (200) in order to facilitate scheduling and to enhance procedure efficiency for medical purposes. Efficiency enables cost containment and adherence to patient medical insurance policies. Furthermore, efficiency insures that hospitals or surgical procedure centers are operating at a desired maximum safe volume, so that the highest number of procedures may be safely performed. The application program interface (202) serves as a means of connecting the application user collected data to fundamental surgical procedure elements: a. surgery boards or displays that display updated data to the user(204); b. a prospective case costing module that enables seamless client accounting (206); c. the case scan intake system that allows the client to facilitate the patient admission process (208); d. the patient application that grants the patient user access to specified client data (210); e. a planning and staffing interface to facilitate employee scheduling (212); and f. a streamline surgical processing system that acts as a communication interface between employees and administrators (214). Importantly, surgical procedures require the resources of many different personnel, all required to be at the same place at the same time, for an expected duration of time, with potentially some or all of those same personnel required for additional surgical procedures on the same day. Accordingly, a principal aspect of the present invention rests with the management of personnel by hospital or surgical center administrators, mindful of the skills, qualifications and experience required to optimally perform any given surgical procedure taking into account a multitude of factors such as cost and patient condition and patient overall health. For example, some personnel may be best suited for a pediatric patient while others more adept at seniors. Also, the staffing levels required within the operating room may be optimized, along with consideration of which employees must drive the furthest, taking into account external conditions like traffic and weather. Moreover, human resources considerations such as overall weekly workload and vacation schedules need to be simultaneously considered according to the present invention.

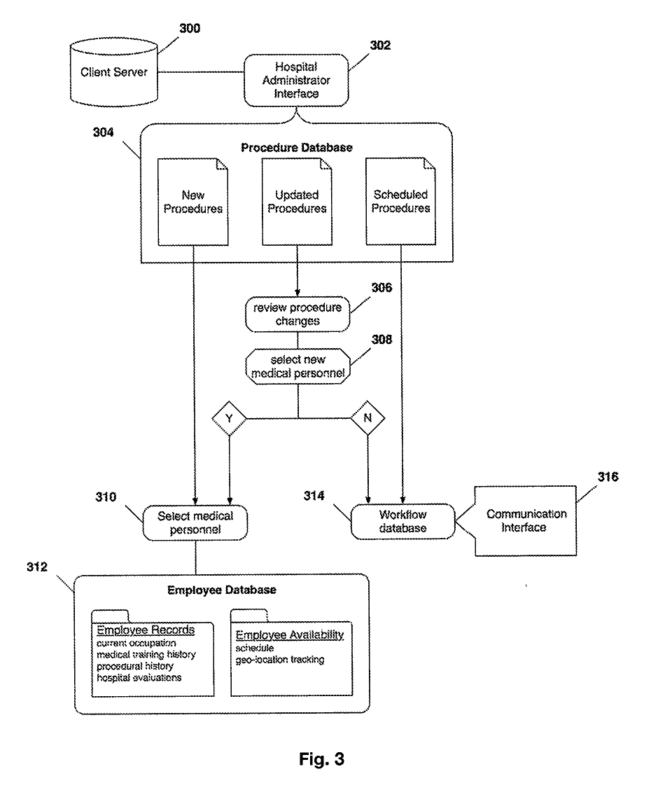

FIG. 3 is a block diagram, which describes the procedure database in the administrator interface according to the present invention. The client or hospital administrator may use the Hospital Administrator Interface (302) to access the procedure database (304) to schedule employees for newly posted procedures.

Also, the administrator may review updates made to procedures and make changes to scheduled employees, and can access the overall workflow database. By this method, the amount of resources can be changed. For example, if a hospital has ten operating rooms and only eight are available because are being updated, the administrator needs to account for what is and is not an available resource. The hospital administrator or surgery center administrator may log into and thereby access the administrator interface (302) data stored on the client server (300). Once the login has been authenticated, the administrator has access to the procedure database (304). The procedure database (304) aggregates and displays all data related to new procedures, updated procedures and scheduled procedures.

When the administrator selects to view data pertaining to new procedures, the administrator can process the data to begin scheduling employees in relation to the presented data. To enable scheduling for a new procedure, the administrator selects the medical personnel option (310) to access the employee database (312). The employee database (312) consists of employee records that contain information such as: current occupation; medical training history; procedural history; and hospital evaluations, as well as listing the availability of employees including key factors such as current schedule and geo-location tracking data. The administrator uses the information in this database to schedule employees for each new procedure. When the administrator selects to view data pertaining to updated procedures, the administrator can then review status updates made pertaining to a specific procedure and determine if there is to be a change in the employee schedule pertaining to that specific procedure (306). If employee changes are to be implemented (308), the administrator accesses the employee database (312) to revise the list of selected employees for the procedure. When the administrator selects to view scheduled procedures, the administrator can access the workflow database (314) and communication interface (316) in order to review procedure data and communicate with the scheduled procedure employees.

By using this system according to the present invention, a hospital administrator may link feedback from the present invention directly into human resources files so that skills and performance during surgery may be tracked, so going forward, optimal surgical teams are formed. For example, a particular nurse may function optimally with one set of doctors, and so forth. Obviously, with the advent of artificial intelligence, many user input and output patterns may be anticipated. In one embodiment, the present invention comprises a method for a computer system to interpret an input from a user and generate a response, comprising receiving a user input, converting the user input into an input array having a plurality of concepts, determining if any of the plurality of concepts in the input array is derived from a root concept, if any of the plurality of concepts is derived from a root concept, replacing each such derived concept with the corresponding root concept, identifying one or more related concepts that relate to the root concept, and generating a multi-dimensional array based on the input array that includes the one or more related concepts, correlating a concept in the multi-dimensional array to a first element in a database, wherein the first element in the database includes a link to a second element in the database, determining a plurality of possible responses to the user input based on the correlation of the multi-dimensional array and the database, and generating a response to the user input. The response may be presented in a textual, audio, visual, or audiovisual format. The response to the user input may be generated based on a selection of one of the plurality of possible responses.

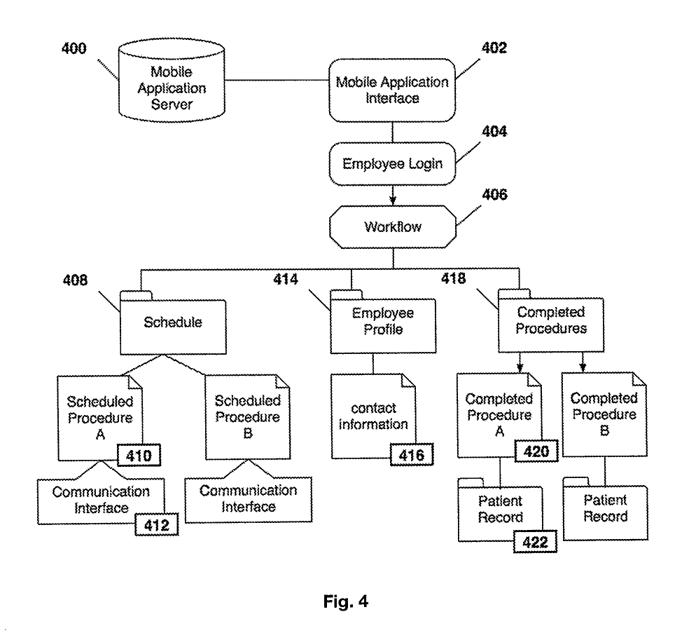

FIG. 4 is a block diagram, which describes the employee workflow interface. By way of reference to FIG. 4, when an employee logs in (404) to access the mobile application interface (402) from the application server (400), the employee can access the workflow interface (406). Employee access to the workflow interface (406) allows the employee to view their scheduled procedures (408), view and edit their employee profile (414) and view all completed procedures (418) as well as post-procedure information. Through the workflow interface, the employee can then access his or her schedule (408) and view detailed information for each procedure (410) that the employee is scheduled for. Each procedure contains a private communication interface (412) that is accessible by the administrator and the scheduled employees of the procedure.

The employee can also access his or her employee profile (414), which contains information included but not limited to employee contact data (416). The employee has the capability to edit and update his or her own contact data (416) if necessary through the employee profile (414) interface function. The employee can also access a record of all completed procedures (418) that the employee has participated in through the workflow interface. Each completed procedure (420) also contains patient records (422) that allow the employee to facilitate patient follow-up once the procedure has been completed.

By way of the present invention, employees of a hospital or surgical procedure center may monitor their work hours and their performance criteria if desired by the system administrator, so that as future surgical teams are built for various surgical procedures, performance criteria may be entered and evaluated to automatically select optimal teams. Furthermore, certain personnel work at various discrete even competing facilities. So, the present invention may be adapted to crowd source medical staff and surgical procedure personnel so that competitive bidding may be employed so that advantageously geo-located professionals may located and asked to bid on certain upcoming scheduled procedures. In that manner, costs may be optimized while quality and safety maintained.

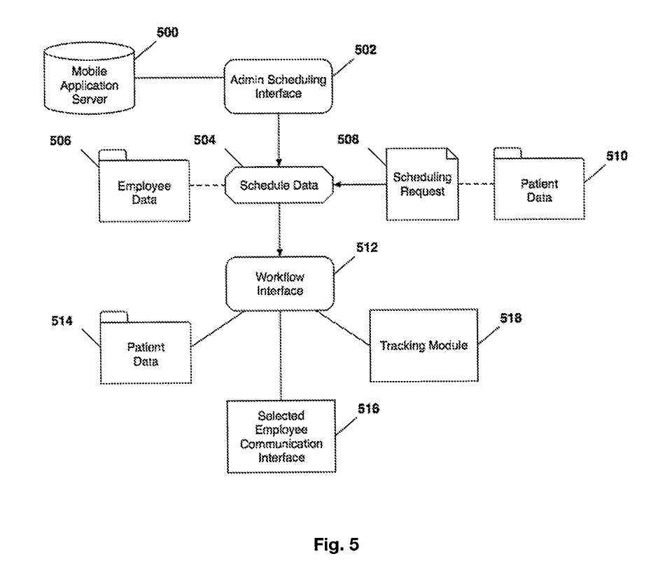

FIG. 5 is a block diagram, which describes the administrator scheduling interface and outgoing workflow interface. The administrator scheduling interface (502) allows the client administrator to view schedule data (504) for procedures and access the workflow interface (512) to view patient data (514), communication between scheduled employees (516) and the tracking module (518) to track employees and patients. The administrator accesses the scheduling interface (502) by logging in through the application server (500). The scheduling interface (502) allows the administrator to view schedule data (504) and select specified employees (506) in relation to said data. Patient data (510) aggregated from the patient intake admissions process is formulated into a specific procedure schedule request (508) that is submitted into the scheduling database. The administrator has to process new procedure scheduling requests (508) in the schedule database (504) and determine how the procedure is to be staffed. Schedule data incorporates all employee data from the employee database (506), this data allows for the administrator to complete the scheduling request. Once the necessary employees have been confirmed for the procedure, the scheduling request is complete. The administrator can then access the workflow interface (512) for each scheduled procedure. The workflow interface allows the administrator to access: patient data records and status (514); the communication interface for scheduled employees (516); and the tracking module (518) that allows for the administrator to use tracking technology to update procedure status.

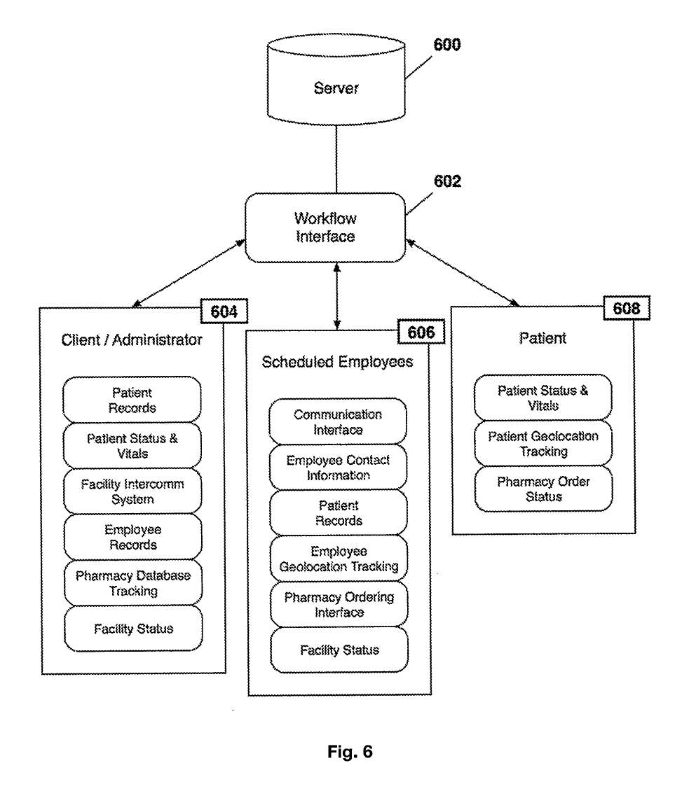

FIG. 6 is a block diagram, which describes the procedure workflow interface. The memory provided by the workflow interface allocates sufficient resources corresponding to the workflow interface options available to the client administrator, employees who are scheduled for the procedure and the patient. The workflow interface (602) serves as the main information aggregator and display module for all application users. The information is aggregated from the data stored on the application server (600) and can be accessed, buffered and displayed through various systems. The application users can be divided into separate groups who may or may not selectively granted specified access levels and are able to view information based on their individual access level. Such access levels may be varied as desired by the system administrator (604). When the client administrator accesses the workflow interface (602), the client administrator (604) is able to view: patient records; patient vitals and status updates; employee records; pharmacy inventory and medication tracking; updated status reports pertaining to the medical facility; and access to the medical facility intercom system. Of course, any multitude of data centers critical to the workflow may be incorporated herein. The administrator has access to the majority of the information on the workflow interface, if not all of it. In addition, limited data may be provided to outside contractors such as the vendors of specialized medical equipment such as hip replacement parts or surgical stents, or outside financial interests such as third party payment interests like health insurance companies or health network interests. In an alternative embodiment, artificial intelligence may be used so that hospital administrators can assess compliance with performance and efficiency requirements and to insure patient safety.

When the scheduled employee accesses the workflow interface (602), the scheduled employee (606) is able to access: the communication interface between other scheduled employees and the administrator; the contact information for the other scheduled employees; patient records, vitals and current status; tracking data for the other scheduled employees; pharmacy ordering capability; and updated status reports pertaining to the medical facility. When the patient accesses the workflow interface (602), the patient (608) can view: current vitals and status; personal geo-location tracking within the facility; and the patient's pharmacy order status. Of course, it is possible for the system administrator to add to or modify any of these permissions.

According to the present invention, displays are typically unidirectional in that they provide information to the viewer. The viewer may be any one of a number of surgical constituents, ranging from the patient and its friends and family, to the surgeons and all those associated with the medical team and facility, including outside vendors. In all cases, the output interface must be readily adaptable for any number of formats such as iOS, Android, Microsoft Surface, HDMI, and any other video monitoring standard. Furthermore, serial and parallel port communication is anticipated, and the ability to store and refresh as often as necessary. Bi-directional communication may be enabled via tablets and touchscreens or “smartboards”, so that display interfaces are often bi-directional according to the present invention. Various video algorithms may be employed along with video compression to enhance the user experience, and as well, cameras may be deployed throughout a hospital facility in order to enhance the ability of the present invention to track patient and surgical procedure assets, along with medical staff personnel and assets. Through the use of video recognition, cameras or other imaging devices may be deployed to track the throughput of a hospital or surgical center. In addition, RFID tags may be deployed so that patient and surgical team assets may be tracked and coordinated with the overall data management schema according to the present invention.

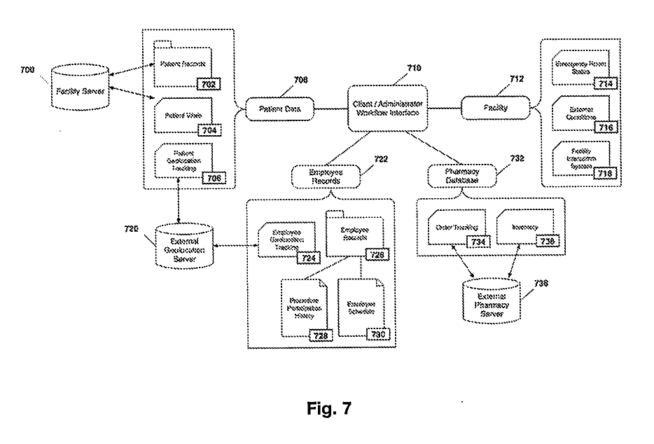

FIG. 7 is a block diagram, which describes client administrator data aggregation for this detailed description of the preferred embodiment. A detailed overview of the workflow interface (710) options available to the client administrator include: patient data (708), records and tracking (706); employee records (722), schedule (730) and tracking (724); pharmacy (732) inventory (736) and order tracking (734); and facility communication and information (712). The administrator accessed workflow interface (710) aggregates all data related to client administration as a means of facilitating the overall management and organization of a medical facility. Of course, it is possible for the system administrator to add to or modify any of these permissions. Patient data that is aggregated includes all patient records and all updated patient vitals that are stored on the facility server. Geo-location based patient tracking data (706) is aggregated from an external geo-location server (720). Employee records data (722) that is aggregated for administrator access includes employee records (726) that contain procedure participation and attendance history (728) for each employee and the overall employee schedule of all procedures (730). Employee geo-location tracking data (724) is aggregated from an external geo-location server (720) and is tracked by various tracking implements.

The pharmacy database (732) accessed by the administrator through the workflow interface (710) grants the administrator access to view the pharmacy inventory (736) as well as track the status of pharmacy orders (734). The pharmacy data is aggregated from the external pharmacy server (738). The administrator can also view data related to the medical facility (712) that includes: the current status of the emergency room (714); external conditions such as weather (716); and access to the medical facility intercom system (718).

In all cases, it is possible for the system administrator to add to or modify any of the permissions associated with any system users. Notably, a principal advantage of the present invention is its ability to monitor, track and provide feedback to each user and administrator. In this manner, a hospital or surgical center administrator may monitor multiple surgical procedures simultaneously, regardless of where the procedure is being performed. In this manner, large hospital chains may have a central administrator who monitors surgical procedures across the globe, and as a result, control costs while maintaining quality control. Fraud mitigation is also a principal advantage of the present invention, and by detecting the performance of all associated personnel, it is possible to optimize procedures and practices going forward.

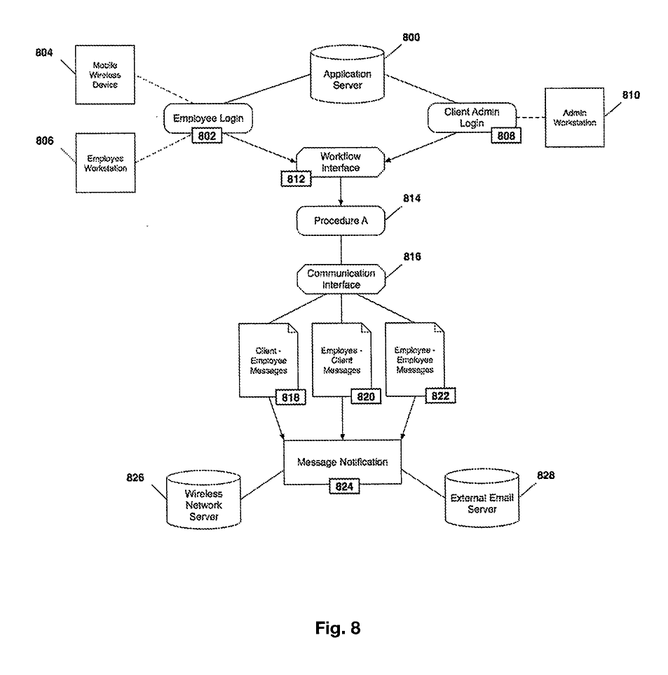

FIG. 8 is a block diagram, which describes the communication interface according to the present invention. In accordance with the preferred embodiment of the present invention, the communication interface module (816) accessed from the workflow interface (812) allows for: the client administrator to send a message to all employees scheduled for the procedure (818); employees to send a message to the client administrator (820); and employees to send messages to other scheduled employees (822). The employee can login (802) to the workflow interface (812) through several methods, including but not limited to a wireless mobile device (804) or an employee workstation (806) within the client facility. The administrator can login (808) to the workflow interface (812) through an administrator workstation (810). Once the administrator or the employee has been authenticated and logged into the workflow interface (812) through the application server (800), the administrator and the employee may select a specific procedure (814) to access the communication interface (816).

The administrator may access the communication interface at any time. In addition, the employees who are scheduled to take part in that procedure may have access as well. The communication interface for scheduled procedure employees and administrators may offer three different communication modules. The first communication module is for handling messages from the client administrator and the employees (818), allowing for the administrator to message either all scheduled employees at once or message a specified employee directly. The second communication module is for handling messages from the scheduled employee to the client administrator (820), allowing the scheduled employee to message the administrator directly. The third communication module is for handling messages among the employees scheduled to participate in a procedure (822), allowing each scheduled employee to message all other scheduled employees or directly message a specific scheduled employee. All messages transmitted through the procedure workflow communication interface notify the intended recipient(s) with a message notification alert (824) that can be processed through the recipient(s) wireless network server (826) or external email server (828).

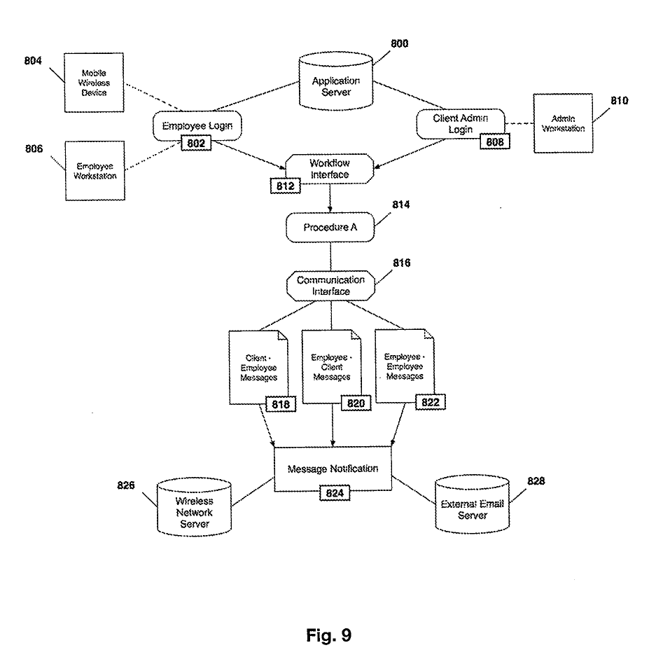

FIG. 9 is a block diagram, which describes the patient profile interface according to the present invention. In accordance with the preferred embodiment of the present invention, the patient interface (910) allows for the patient to view data related to a specific procedure (920). When the patient enters his or her login information (904) into the application server (900), the patient identity is verified (906) through the client administrator security database (908) to ensure confidentiality compliance. Once the patient has been verified by the administrator, the patient is authorized to access the patient interface (910). Within the patient interface, the patient can access his or her personal pharmacy order information and updated order status through the pharmacy module. Also within the patient interface (910), the patient can access a list of his or her scheduled procedures (920). The patient selects the scheduled procedure (922) to view: procedure information (924); the list of scheduled employees (926) and their subsequent employee profiles (928); the current status of the procedure (930); and the admin messaging interface (932) that allows for the patient to send and receive messages to the administrator. The patient can also access their pharmacy data (912) through the patient interface (910), whereby the patient can view their personal order list (916) and order status (918).

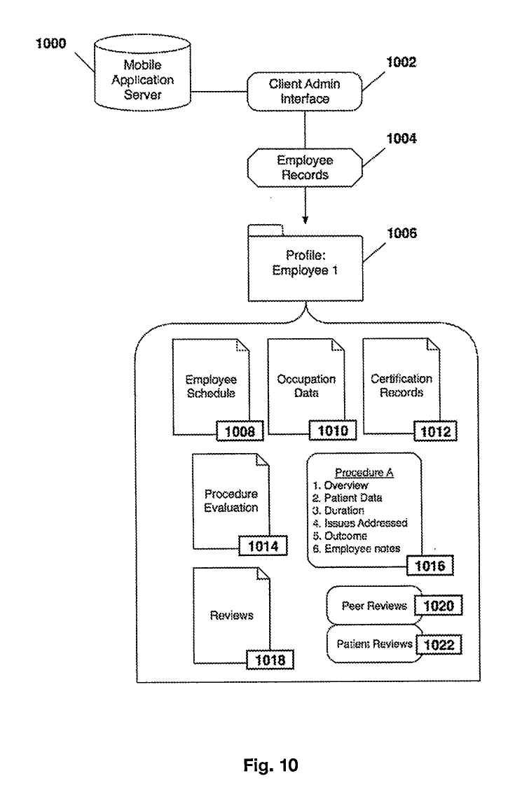

FIG. 10 is a block diagram, which describes the client administrator employee profile interface according to the present invention. In accordance with the present invention, the client administrator (1002) is able to access individual employee records (1004) that display the employee's schedule (1008), occupation data (1010), employee certification summary (1012), evaluations for each procedure (1014) and reviews from other employees and patients (1018). When the administrator logs into the client administrator interface (1002) through the application server (1000), they are able to access a database containing all medical facility employee records (1004). When the administrator selects a specific employee profile (1006), the administrator has the option of viewing: the schedule of the selected employee (1008); employee occupation data (1010); employee certification and training records (1012); employee reviews (1018) submitted by other employees (1020) and patients (1022); and procedure evaluations (1014). Each procedure evaluation record (1016) contains the relevant data for each procedure that the employee has participated in, and includes information such as: procedure overview; patient data; procedure duration; issues addressed during the procedure; the outcome of the procedure; and employee notes.

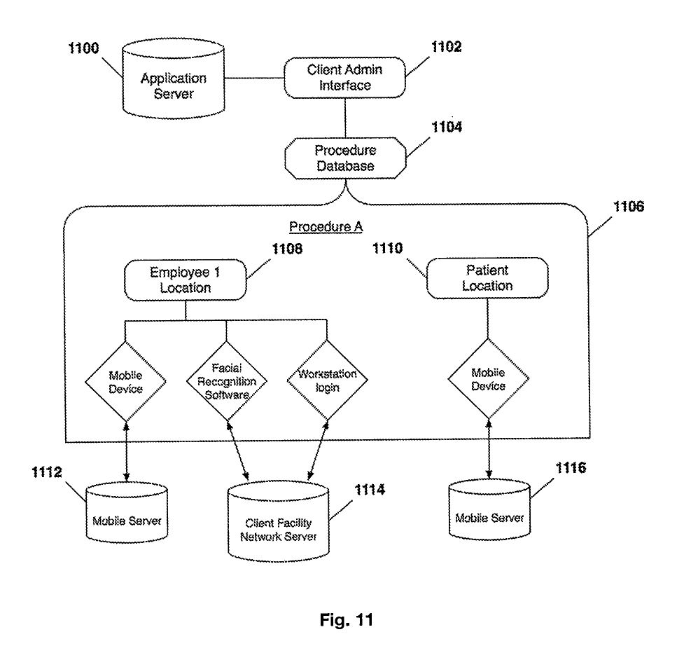

FIG. 11 is a block diagram, which describes the client administrator geo-location monitoring system according to the present invention. In accordance with the preferred embodiment of the present invention, an overview of the geo-location monitoring system is made available to the client administrator (1102), allowing the client administrator to track patients (1110) using a configured mobile tracking device and track scheduled employees (1108) for a procedure using: a configured mobile tracking device; facial recognition software set up in designated facility locations; or employee login at designated facility workstations. Through the administrator interface (1102), the administrator selects a specific procedure within the procedure database (1104). One of the components available for the administrator may include the ability to track both the employee and the patient for every procedure. Employee tracking data (1108) is aggregated from the employee's mobile device, facial recognition or other biometric recognition software or logging into a facility workstation. Mobile tracking data is obtained from the mobile server (1112), while recognition sensing and workstation login data is obtained from the medical facility network server (1114). The employee tracking data is then displayed for the administrator within the procedure database (1104). Patient location data (1110) is aggregated from a mobile device that is worn by the patient or attached to an apparatus that transports the patient within the facility, such as a wheelchair or a hospital bed. Patient mobile device tracking data is obtained from a mobile server (1116), and is displayed for the administrator within the procedure database (1104).

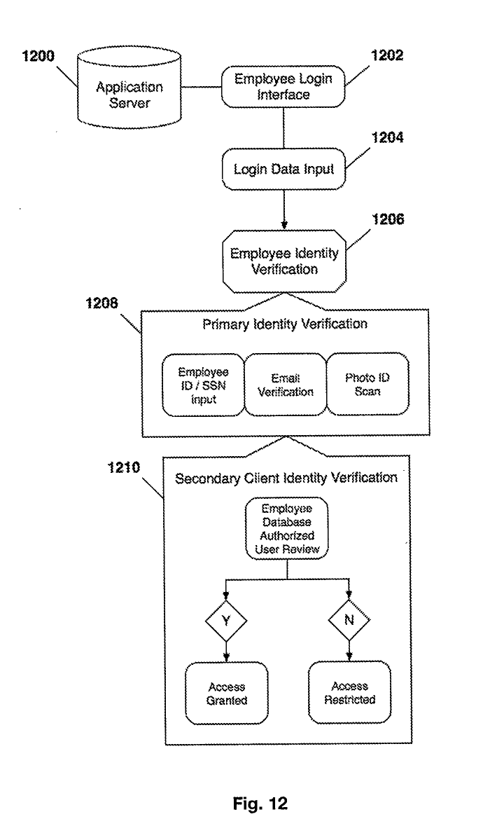

FIG. 12 is a block diagram, which describes the employee identity verification interface according to the present invention. In accordance with the preferred embodiment of the present invention, a security module that uses primary verification (1208) and secondary verification (1210) is implemented to authorize access for employees during the initial login sequence (1204). When the employee initially enters his or her login data (1204), the employee must be verified (1206) through an identity verification system comprising two steps. First, an employee must enter an employee number or social security number; verify the login information through an external email server; and scan his or her photo identification into the application server using a mobile scanner application. And a second identity verification (1210) requires that the administrator review the initial login attempt and verify the employee as being a valid employee. If the employee completes both primary (1208) and secondary verification (1210) successfully, the employee is granted employee user access to the application interface. If the employee does not complete the primary and secondary identity verification process, then employee user access is restricted or denied.

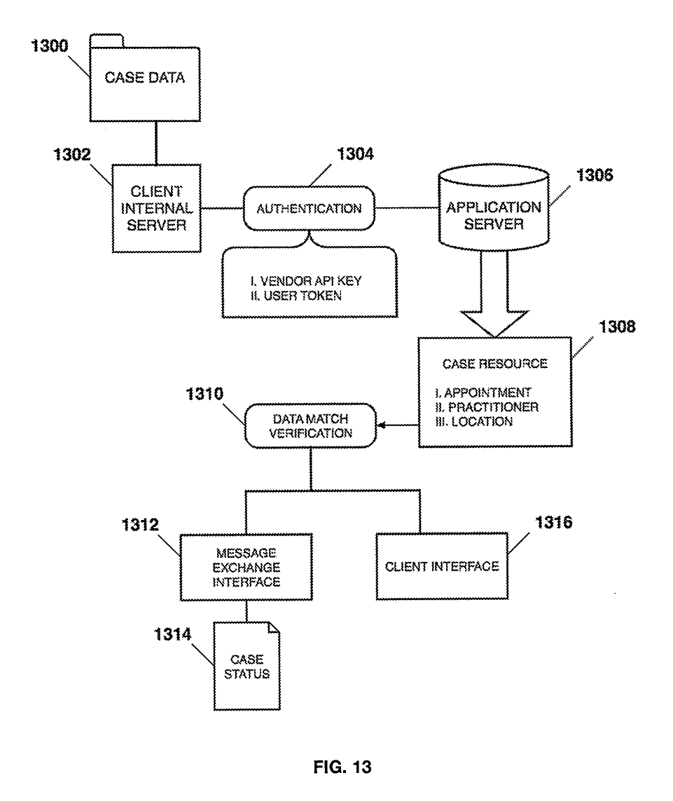

FIG. 13 is a block diagram, which describes inbound data integration between the client's internal server and the online application server. According to the present invention, the online application provides surgical scheduling and online data coordination client case management. Each client may use the same or different vendor provided software system that can store and manage stores and manages case data. These cases and their subsequent updates need to be uploaded to the online application server (1306) as inbound flow. The client will interact with the online application to view, manage, and coordinate these cases. Basic information flow is one-way message drop from Client's Internal Server (1302) to the application server. Each client is expected to transmit case data (1300), either on a real time basis or at a regular interval. Updates to the case data are based on the client's internal system. In addition to the case data resource, “appointment”, “practitioner” and “location” resources are also supported (1308). Authentication (1304) will be based upon API keys and tokens provided by the application system server for each client. (1306) The API key provided will be used to authenticate and return a user token. This token will expire within 24 hours of creation. All pertinent parameters for case data (1300) are formatted in JavaScript Object Notation (“JSON”) documents using a Portable Operational Support Terminal (“POST”) system. Subsequent case data (1300) updates on these cases are uploaded to the application server (1306) by sending the same payload with updated values on a POST system. All Fields, such as the full case data payload and new values, are expected to be present within the case data resource (1308). The online application system (1306) must get initial resource files in encrypted comma separated values (“CSV”) or using the POST method to populate “practitioner” and “location” resources, to lists all Physicians and Room IDs. This data is verified (1310) before it is available on the message exchange (1312) and client interface (1316). Other fields such as patient names are not initially required and can be populated through the application system. upon receiving new case data, If the Physician Name or Room ID doesn't match verification parameters, the case data (1300) message will be rejected. For each connection, it is expected to have reasonable amount of customization. Any part of protocol and data format can be formatted within the client server (1302) for scheduling and coordination.

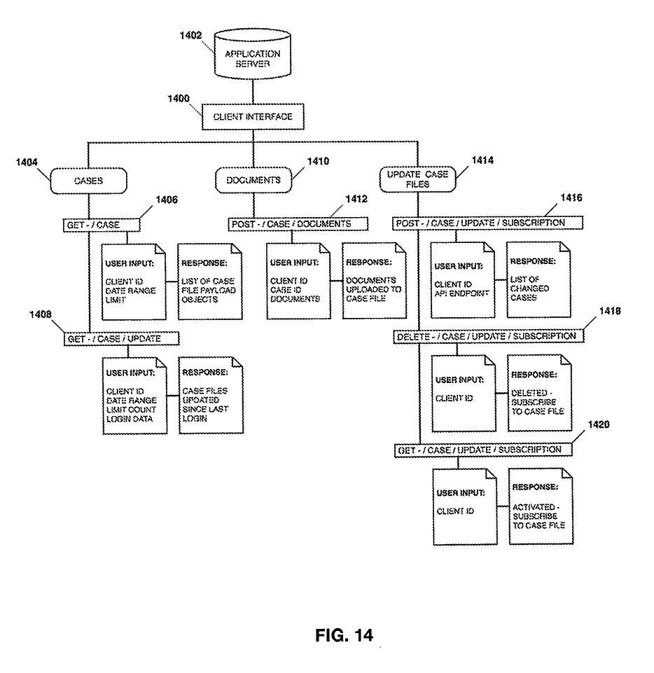

FIG. 14 is a block diagram, which describes the outbound data flow within the client interface. In accordance with the preferred embodiment of the present invention, once case data has been successfully uploaded to the application server (1402) as inbound workflow, the client can use the client online client interface (1400) to retrieve case data as well as subsequent updates. The client can retrieve case data (1404) within the Cases Application Program Interface (“API”). This API returns a list of cases (in JSON format) that matches the time range specified in the request (1406). If a limit value X is specified, only X number of records will be returned; repeating the same request will return a set of X values until all case data is displayed. The update API (1408) returns any cases that have been updated since timestamp T. Also, there are many fields within the present invention that are currently not exposed to the API, such as case team member and vendor requests. The changes only track the fields defined for the case data JSON document. The update API enables a vendor system to get all the data from the application server (1402) and to facilitate client synchronization. Furthermore, if a case is updated within the vendor system, it can issue case API to update the case. The Documents API (1410) is for uploading any type of document (1412) to the application server (1402) that is associated with a case file. Binary documents are typically converted to base64 string and encoded in utf8 format. The present invention can also consider sending the vendor on case changes in real time. This requires the vendor to expose their public API endpoint so application server can receive the updated case data. (1414). The initial authentication to the vendor API is negotiated on a case by case basis. The present invention can assume the session is established and the base URL to the vendor API endpoints is known. The vendor system can issue a subscription interest to changes to any client data file (1416). A URL will be constructed by using the authenticated session with the callback API endpoint. Then the application server will issue a request call to this API with updated cases (1416). The interface also allows for the option to delete the update subscription (1418), which is automatically timed out at midnight Pacific Time. A request to re-establish subscription status (1420) must include the client ID number, whereby the API endpoint is registered and the subscription is re-established.

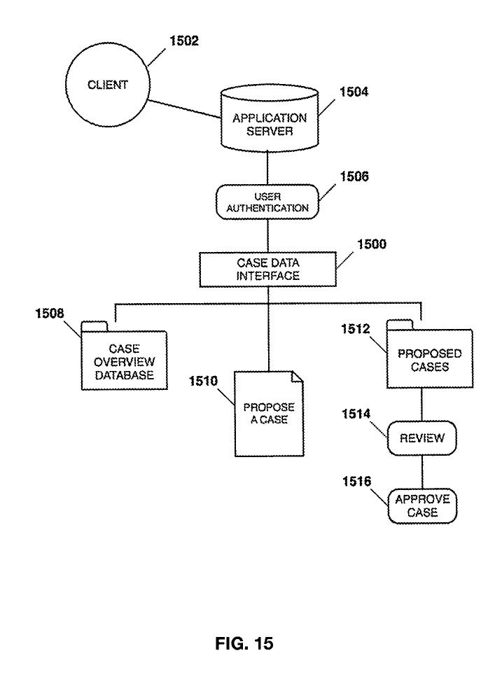

FIG. 15 is a block diagram, which describes the case proposal functionality for the client system. In accordance with the preferred embodiment of the present invention, the client (1502) is authenticated (1506) within the application server (1504) of the present invention to access the case data interface (1500). The home screen of the client interface is an overview of all client cases uploaded to the client database (1508). The client can submit a direct case proposal using the propose a case function (1510) within the interface. The client can also access all current proposed cases (1512) whereby the proposed case is reviewed (1514) and potentially approved by the designated client administrator.

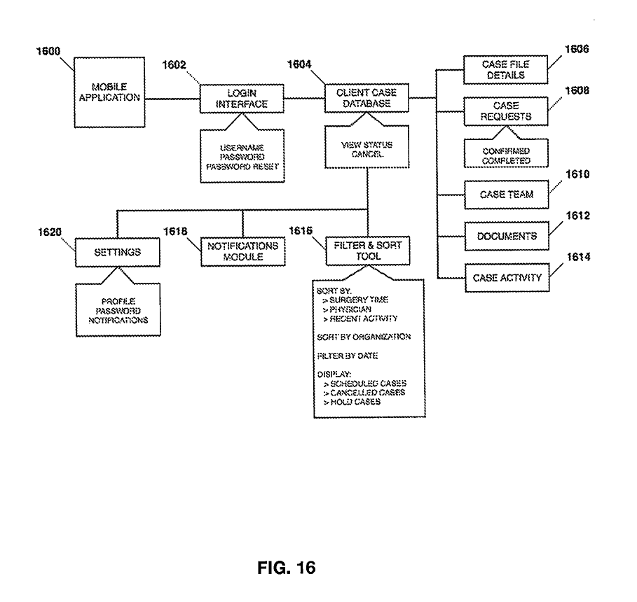

FIG. 16 is a block diagram, which describes the mobile application features available to the client for accessing case data files. In accordance with the preferred embodiment of the present invention, the mobile application (1600) consists of the following display components for mobile case data retrieval by the client. The client can access the mobile case database (1604) once they are successfully logged in (1602) by entering a username and password or securely resetting the password. The database is navigated through five overview modules. Selection of the case file details (1606) displays the full data record for a specific case file. The case request module (1608) displays all cases that are proposed and can be confirmed or deleted. The case team module (1610) allows for the client to view and enable communication with other scheduled client users who have been assigned to a particular case file. Using the documents module (1612), the client can directly upload all necessary documents related to a case file. The case activity module (1614) serves as a record all case data file events or updates in real time. The (1414) case file database has a filter and sorting functionality (1616) whereby the client can sort case files. The client can also access case files using selected parameters in order to facilitate the process of looking for a specific case file. The notifications module (1618) enables the client to email or instantly message other members of the organization or case team, and all messages can be read, forwarded, archived or deleted. The settings module (1620) is for setting up and updating the individual user profile on the mobile application, as well as customizing mobile related integration preferences such as app related alerts permissions.

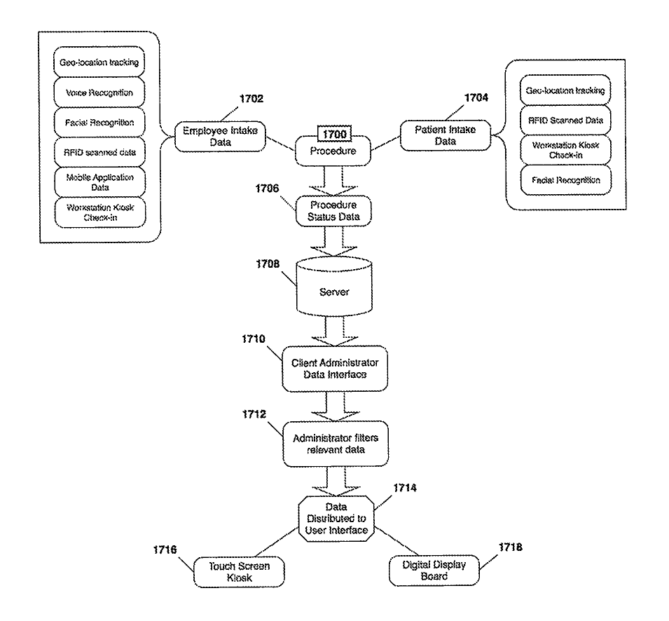

FIG. 17 is a block diagram which describes the aggregation of data that is displayed within the user interface according to the present invention. In accordance with the preferred embodiment of the present invention, data is collected from the employee (1702) and the patient (1704) during the procedure (1700) and is used as a means of providing real-time procedure status updates (1706) within the user interface. During the procedure, data collected from the employee (1702) can include: geo-location tracking of the employee within the medical facility; employee voice recognition and voice recording; employee facial recognition; portable or wearable Radio Frequency identification (RFID) scanned data; data collected from the employee's configured mobile device; and data input from the employee at a designated work-station kiosk. Data collected from the patient (1704) can include: geo-location tracking of the patient within the medical facility; portable or wearable Radio Frequency identification (RFID) scanned data; data input by an employee regarding the patient at a designated work-station kiosk; and patient facial recognition.

All data transmitted during the procedure (1700) serves as procedure status (1706) monitoring information that is stored on the application server. The administrator has access to this data by logging in to the administrator data user interface (1710). The administrator can then analyze and filter data (1712) relevant to the status of the procedure. The administrator selects the specific data that is to be distributed and displayed on the user interface (1714). The selected procedure status update data can be accessed through interactive platforms including but not limited to digital display boards (1718) and touch screen user interface kiosks (1716) located within the medical facility.

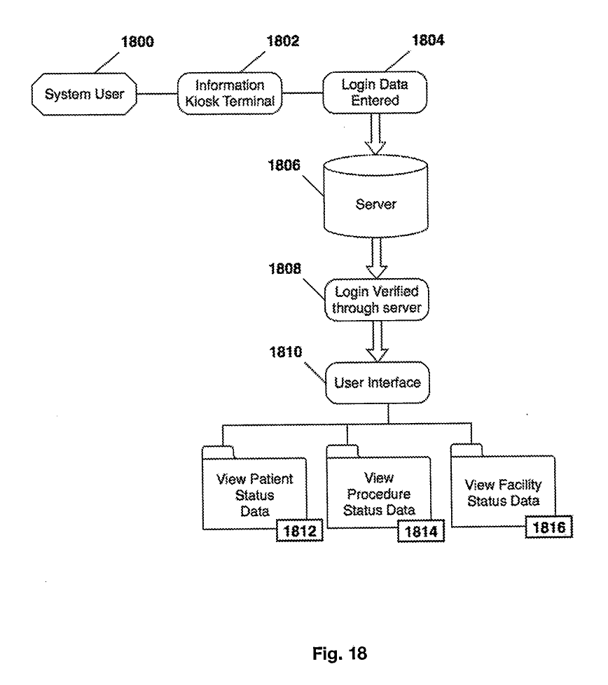

FIG. 18 is a block diagram which describes the user interface when accessed by the system user according to the present invention. In accordance with the preferred embodiment of the present invention, the application user interface (1810) can be accessed through an interactive platform such as an information kiosk terminal (1802) located within the medical facility. Once the user (1800) has entered the login data (1804), the data is transmitted to the application server (1806) and the login is authenticated (1808). If the authentication is successful, the user is granted access to the application user interface (1810), where the user can view data pertaining to: patient status (1812); procedure status (1814); and facility status (1816).

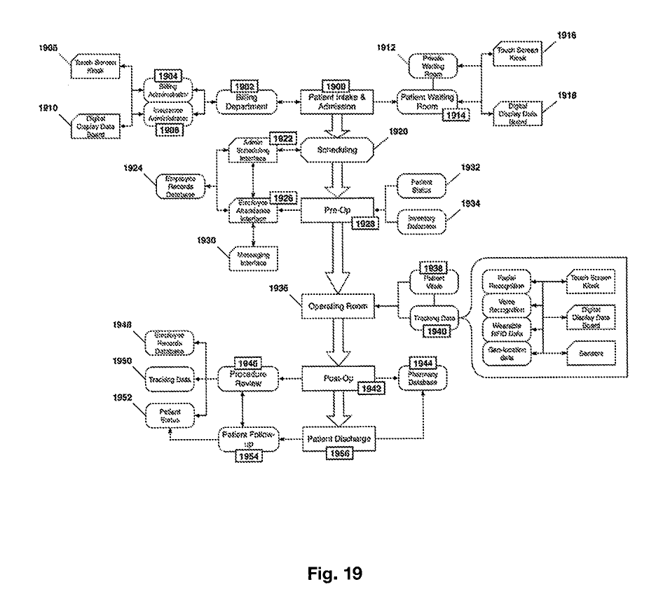

FIG. 19 is a block diagram, which describes the overall user interface according to the present invention. In accordance with the preferred embodiment of the present invention, all data related to the medical facility client administrator, medical facility client employees, and the patient is aggregated and implemented in the application user interface. Data aggregation begins with the patient admission and intake process (1900). The patient intake administrator can utilize the user interface to send and receive data with the client billing department (1902). The billing department comprises of various sub-departments including but not limited to a billing administrator (1904) and an insurance administrator (1906). Each of these sub-departments interact with the application user interface using interactive platforms such as touch screen kiosk (1908) terminals and digital display boards (1910), to send and receive data. The patient intake administrator can also utilize the application user interface to interact with the patient waiting room (1914) by displaying relevant information to patients and their relatives (1912) using user touch screen kiosks (1916) and digital display boards (1918).

Once the patient has been admitted through the intake process, the patient intake administrator logs in to the administrative portal of the user interface to access the procedure scheduling interface (1920). The administrator uses the scheduling interface (1920) to schedule available employees (1922) to attend to the admitted patient. The administrator reviews employee schedules (1922) and the employee records database (1924) to optimize the employee scheduling process through real-time updated information.

Once the procedure schedule is confirmed by the selected employees and the administrator, the application user interface can be utilized by the selected employees and administrator at the pre-op phase (1928). The administrator and selected procedure employees can access a private communication portal (1930) to facilitate messaging between the procedure employees and the administrator. The user interface can be used by the administrator and employee to get real time updated information on the status of the patient (1932) such as patient vitals (1938) that are continuously updated, monitored and stored on the application server. The user interface also stores and displays data from the medical facility inventory database (1934), allowing the employee and administrator to confirm that the supplies necessary for the procedure are available.

After the necessary pre-op procedures are complete, the user interface collects and distributes data to and from the administrator, employee and patient for the duration of the scheduled procedure. Selected employees can share data with the administrator through the user interface in a specific location of the medical facility, such as an operating room (1936). The specific location of the procedure within the medical facility has the capability to collect data that includes but is not limited to: patient vitals data (1938); tracking data (1940); patient and employee facial recognition; patient and employee voice recognition; wearable Radio Frequency Identification (RFID); and patient and employee geo-location data within the medical facility. This data can be collected using devices such as sensors placed within the medical facility to detect specified criteria and touchscreen kiosk terminals that allow employees to check in and enter procedure updates in real time. This data is transmitted and stored in the application server and can be accessed through the user interface. The user interface can display this data on digital boards and touch screen terminals placed within the medical facility.

Once the procedure is completed, the status on the user interface is updated to post-op (1942), allowing the employee and the administrator to input and access data that reviews the procedure (1946). Procedure review data can comprise of: data distributed to and from the employee records database (1948); procedure tracking data that is aggregated and analyzed (1950); and updated patient status data (1952). Once the patient is discharged (1956) from the medical facility, the user interface provides patient status records and pharmacy database records (1944) to the administrator and employee to optimize patient follow-up (1954).

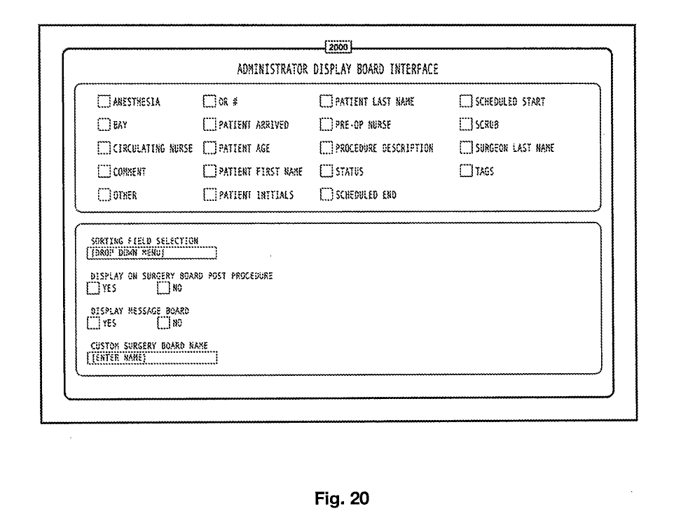

FIG. 20 is a diagram which describes the Administrator Display Board Interface according to the present invention. In accordance with the preferred embodiment of the present invention, the administrator has full access to all data aggregated by the user interface, and can select the data that is to be shared with other viewers of the user interface. The administrator user interface screen (2000) is displayed on an administrator workstation monitor, and allows the administrator to specify the data that will be posted on digital display boards to be viewed by patients and employees.

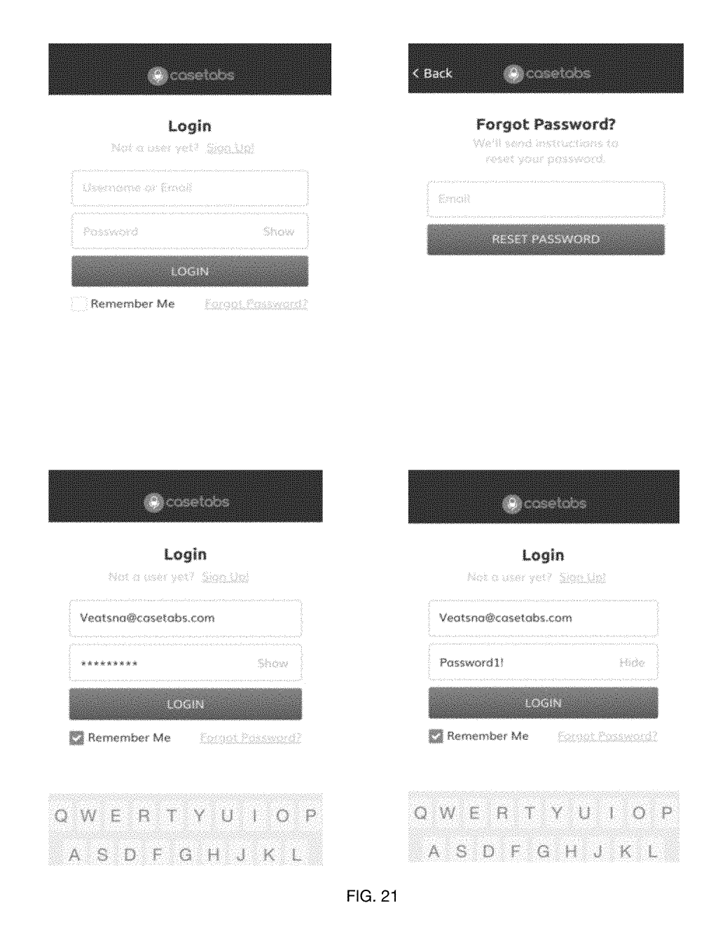

FIG. 21 depicts an example of an embodiment according to the present invention, which describes the login parameters for the application interface. In accordance with the preferred embodiment of the present invention, valid user login credentials are required to access the application interface. The user must enter a valid username or email address as well as the corresponding password in the login screen. within the login screen, the user has the option of showing the entered password to confirm that the password has been entered correctly. The user also has the option to reset his or her password by selecting the forgot password function, whereby the user must enter a valid email address to retrieve password data.

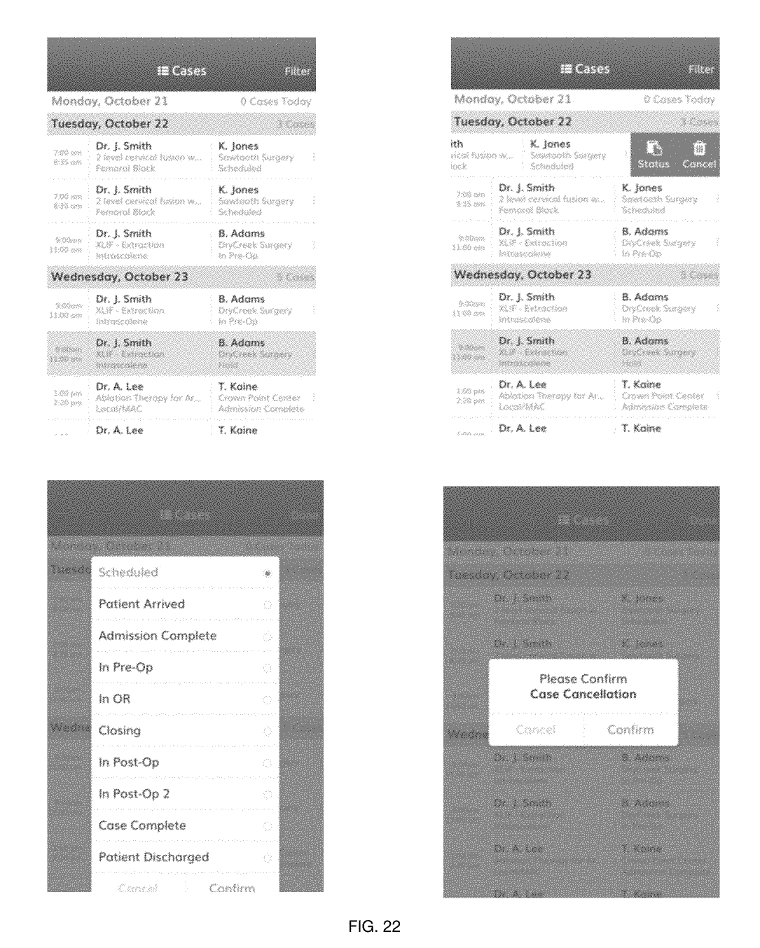

FIG. 22 depicts an example of an embodiment according to the present invention, which describes the case list data overview of the application interface. In accordance with the preferred embodiment of the present invention. The case list overview screen shows all case list data available to the user, whereby each case can be selected, prompting the user to view the status of the selected case or cancel the selected case. In order to cancel a selected case, the user must confirm cancellation in a second dialog screen. The user can also update the status of the case if there is a status change by selecting from a list of status options and confirming that the status has been updated.

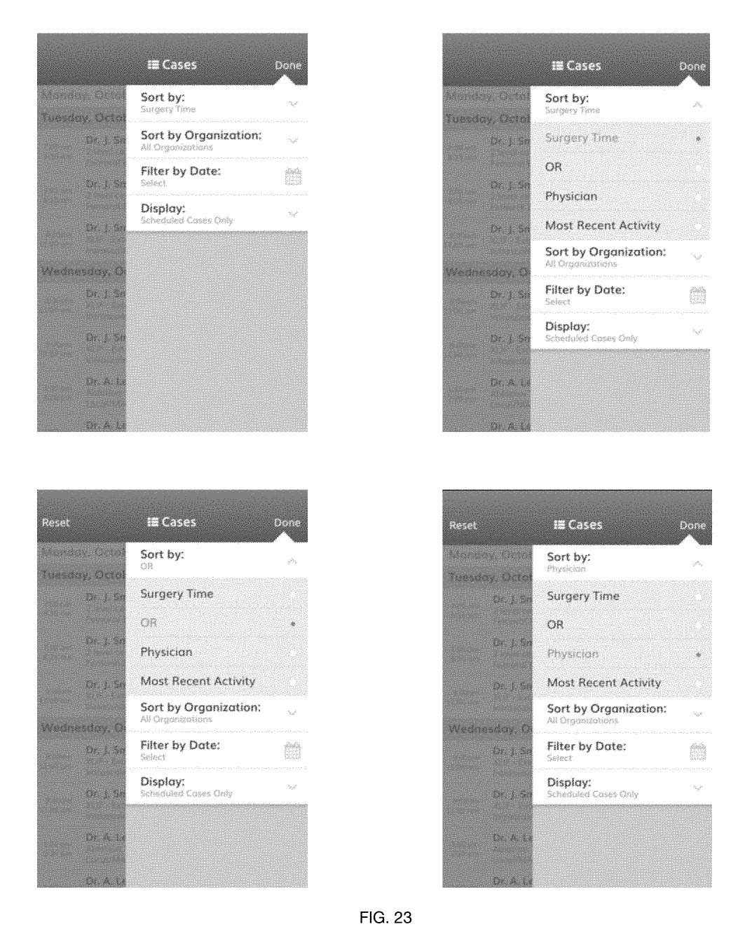

FIG. 23 depicts an example of an embodiment according to the present invention, which describes the filter and sort functionalities of the application interface. In accordance with the preferred embodiment of the present invention, the user can filter the case list data overview through a set of filtering options from the drop down menu that include sorting case list data by: scheduled surgery time; physician assigned to the case; or most recent activity.

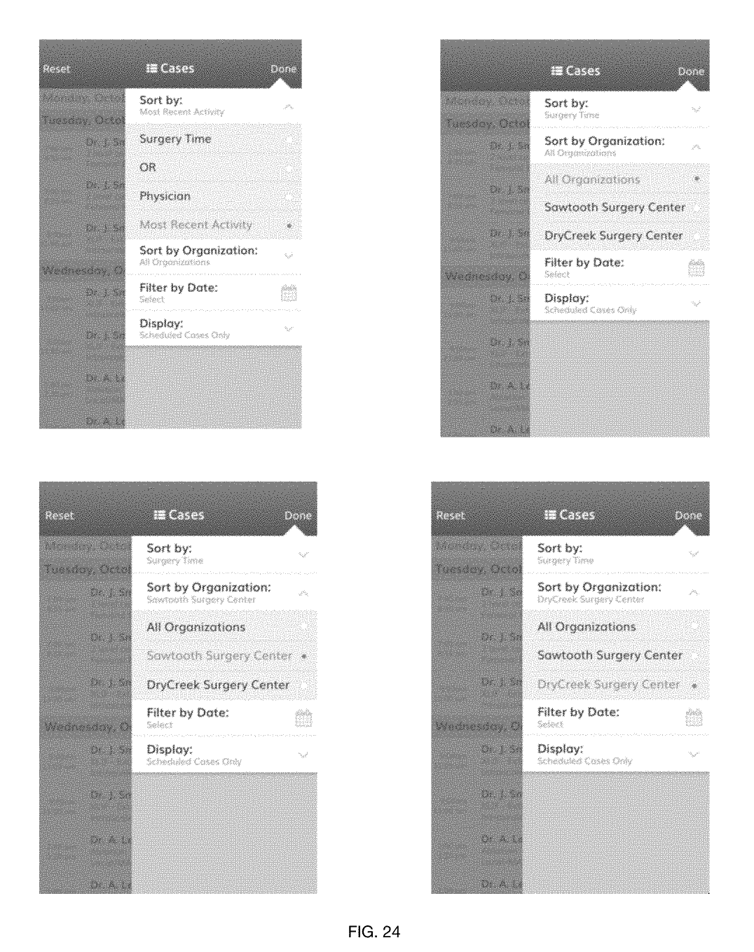

FIG. 24 depicts an example of an embodiment according to the present invention, which describes the filter and sort functionalities of the application interface. In accordance with the preferred embodiment of the present invention, the user is also able to filter the case list data overview by the different organization that the case may be assigned by, if there are multiple organizations associated with the client.

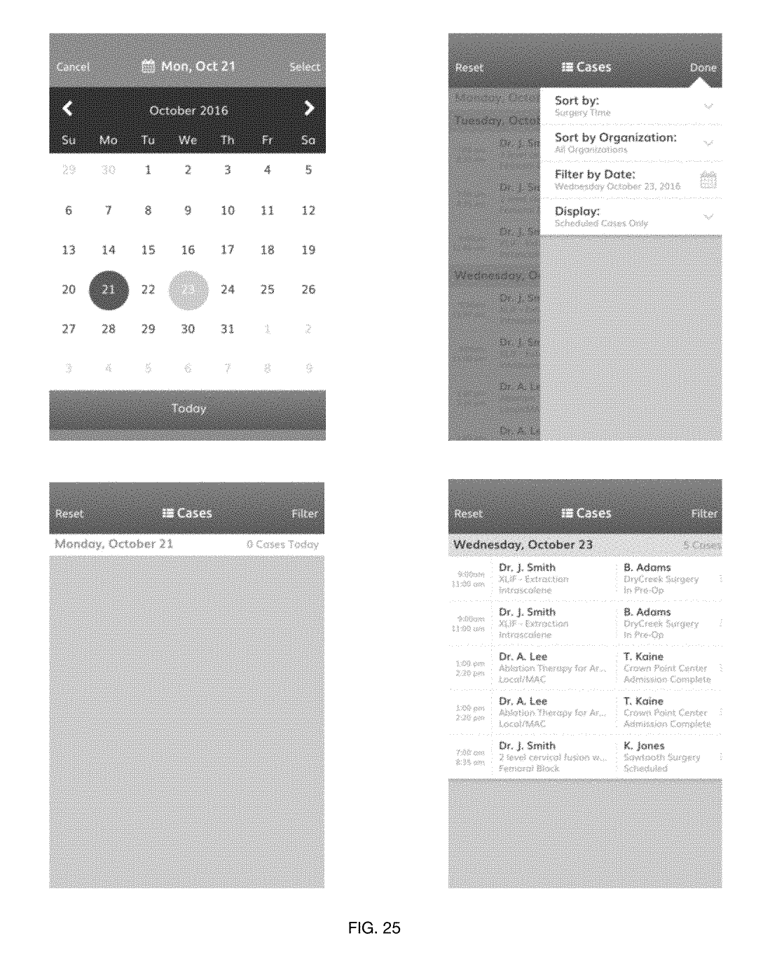

FIG. 25 depicts an example of an embodiment according to the present invention, which describes the filter and sort functionalities of the application interface. In accordance with the preferred embodiment of the present invention, the user can filter case list data by the date of each case, whereby the user selects a specific date or a range of dates on a calendar screen.

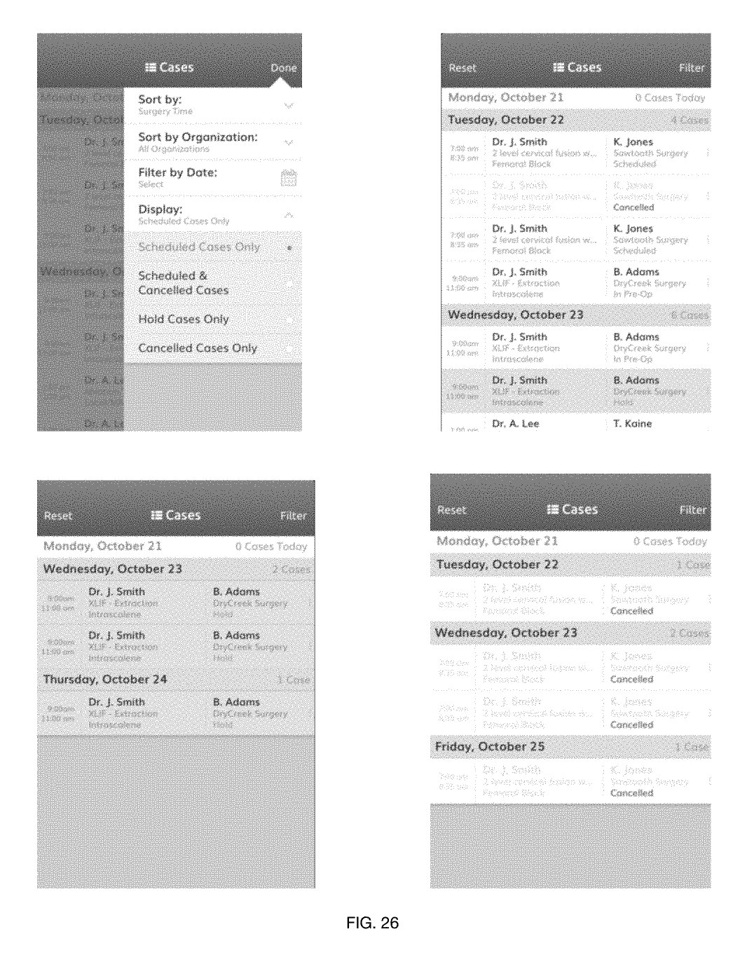

FIG. 26 depicts an example of an embodiment according to the present invention, which describes the display mode of the application interface. In accordance with the preferred embodiment of the present invention, the user can also display cases based on case data, such as selecting the option to display scheduled cases only. Other options for displaying case data include displaying both the scheduled and cancelled cases; display only the cancelled cases or only the cases that are marked as being on hold.

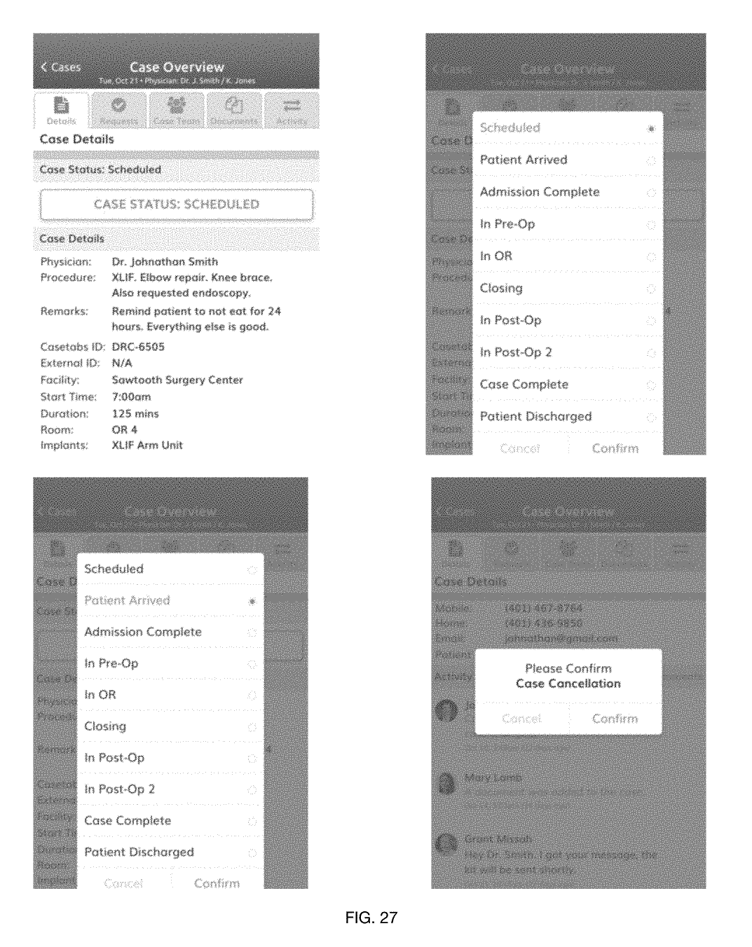

FIG. 27 depicts an example of an embodiment according to the present invention, which describes the details functionality of the application interface. In accordance with the preferred embodiment of the present invention, the user can select a specific case within the case list data overview to view all details pertaining to the selected case. The details screen of each case also allows the user to change the status of the case as well as cancelling the case.

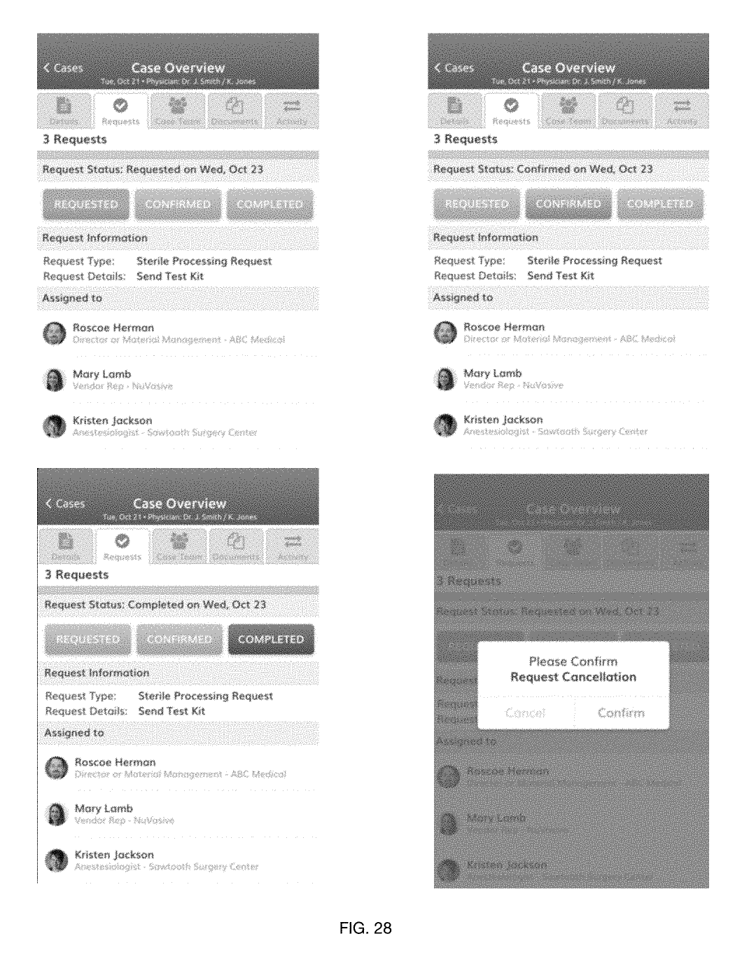

FIG. 28 depicts an example of an embodiment according to the present invention, which describes the requests function of the application interface. In accordance with the preferred embodiment of the present invention, the user can also access case requests within the case list data overview screen. The user selects a case request to review the selected case details. Within the case request details screen, the user can also mark if the request has been confirmed or completed. The user can also cancel the case request through the request detail screen.

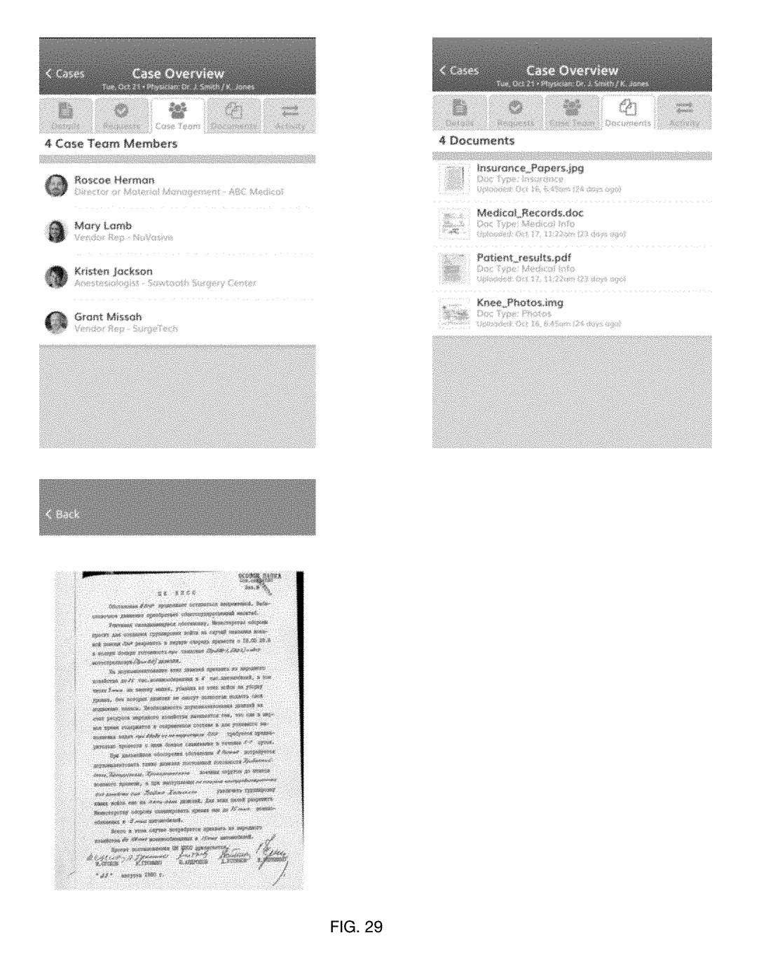

FIG. 29 depicts an example of an embodiment according to the present invention, which describes the case team and documents overview of the application interface. In accordance with the preferred embodiment of the present invention, each case list detail screen also allows the user to view other team members assigned to the case, by selecting the case team tab. The user can also view any documents attached to the case by selecting the documents tab.

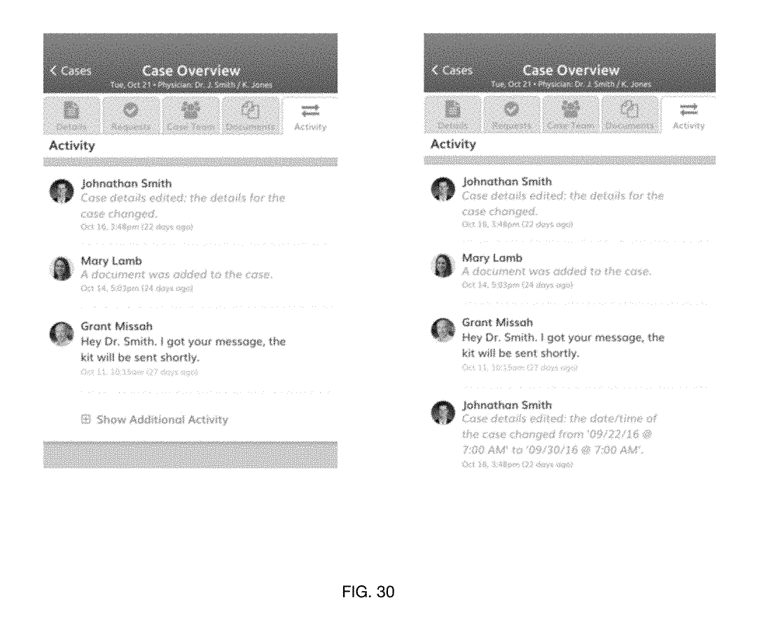

FIG. 30 depicts an example of an embodiment according to the present invention, which describes the activity overview of the application interface. In accordance with the preferred embodiment of the present invention, each case detail screen contains an activity tab that allows the user to view all prior activity for the history of the case file. Each activity shows the user that updated the case as well as the timestamp for the update in chronological order.

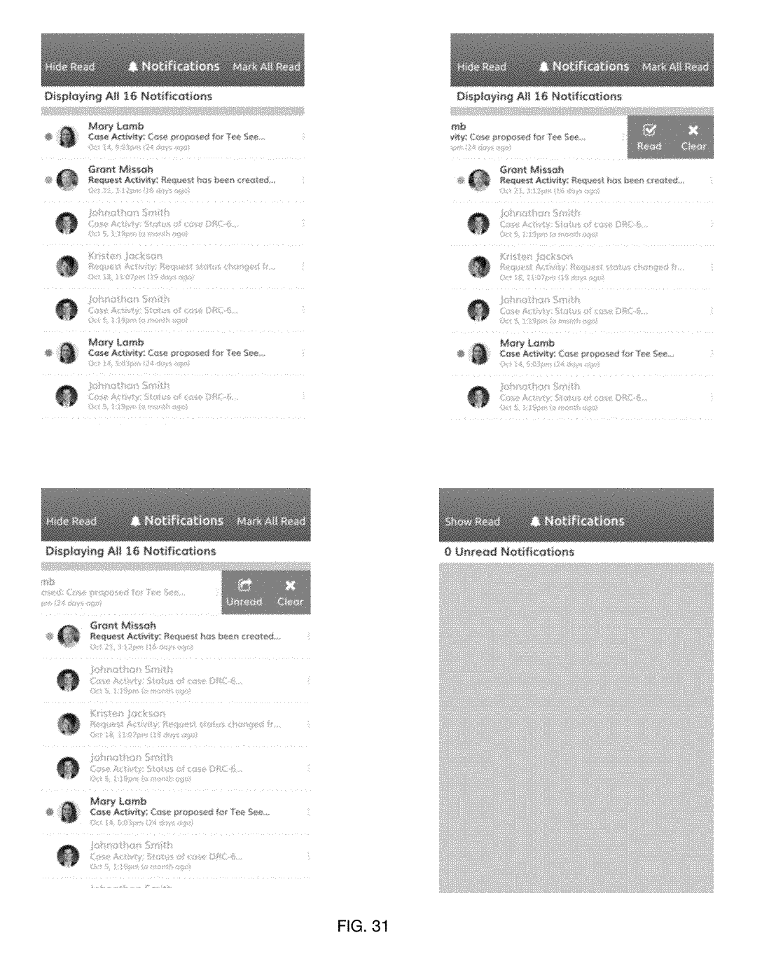

FIG. 31 depicts an example of an embodiment according to the present invention, which describes the notification functionality of the application interface. In accordance with the preferred embodiment of the present invention, the application interface contains a notification function that stores all user notification messages from other application users. Unread notification messages are marked for the user. The user can select each message and is given the option to mark the notification message as read or clear, whereby the message will be deleted. If the user selects a previously read message, an option to mark the message as unread is also given.

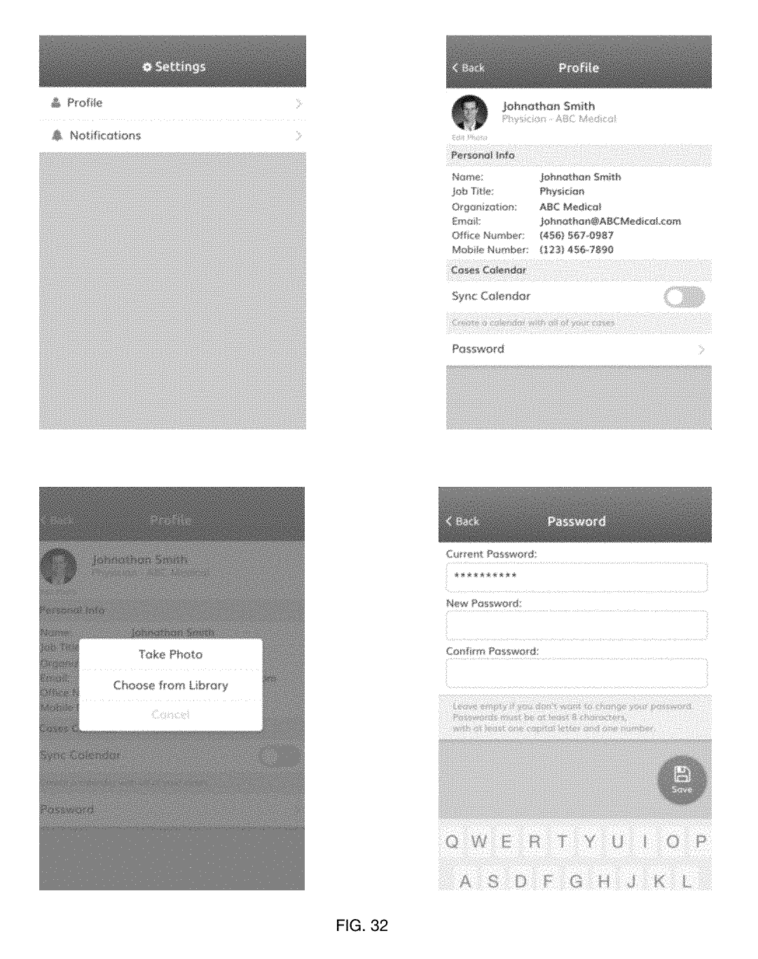

FIG. 32 depicts an example of an embodiment according to the present invention, which describes the settings functionality of the application interface. In accordance with the preferred embodiment of the present invention, the user can access the settings module of the application interface to update the user profile or change the application notification settings. By selecting the profile option within the settings module, the user can view his or her displayed application profile and upload a photo from the user's device to the profile. Through the profile option, the user can also update his or her login password.

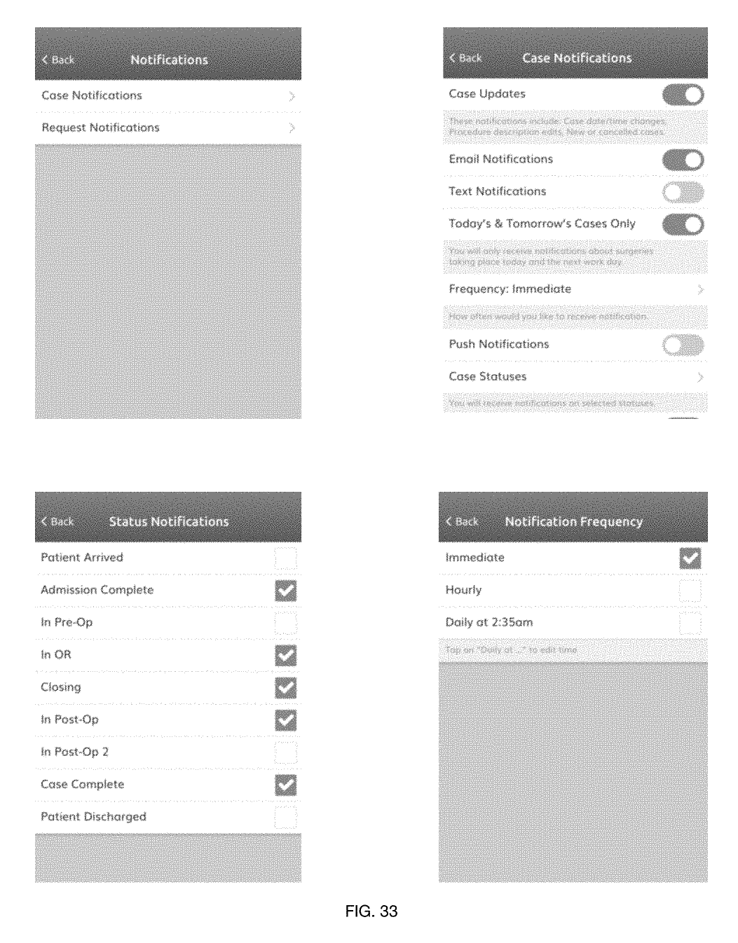

FIG. 33 depicts an example of an embodiment according to the present invention, which describes the settings functionality of the application interface. In accordance with the preferred embodiment of the present invention, the user can update application notification preferences through the settings module, for case related notifications as well as request related notifications. Case notification settings can be turned on or off specifically for: case updates; email notifications; text notifications; and notification reminders for upcoming cases. The frequency of notifications can be changed to three settings: immediate, hourly; or daily. Push notifications for cases can be switched on or off and the user can specify notifications for cases that are marked with a specific status.

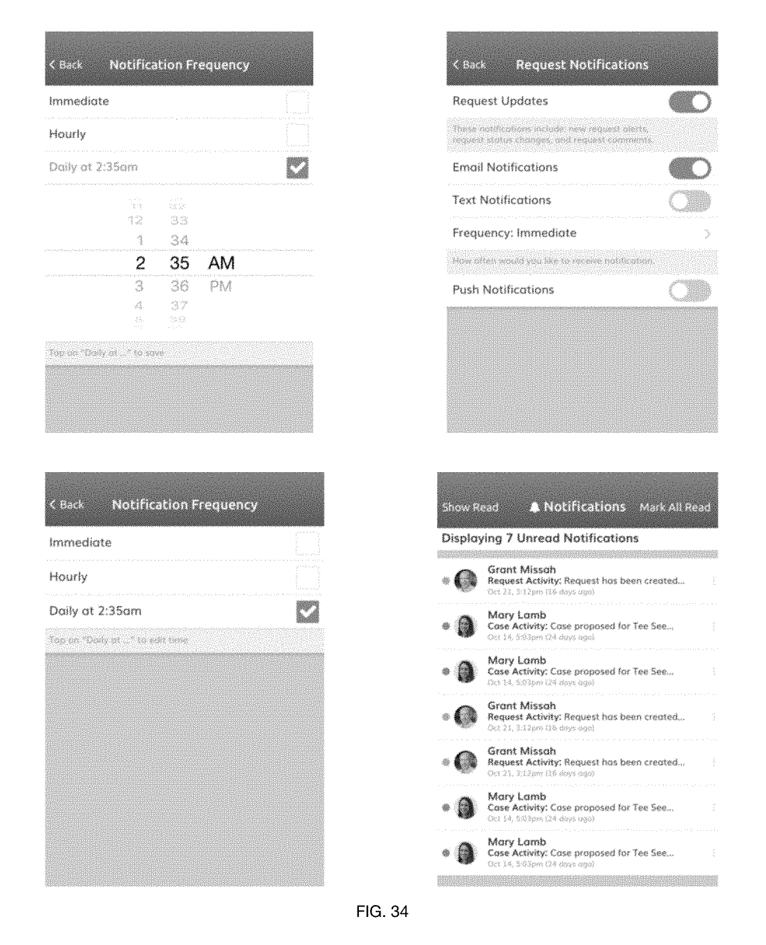

FIG. 34 depicts an example of an embodiment according to the present invention, which describes the settings functionality of the application interface. In accordance with the preferred embodiment of the present invention, the user can update application notification preferences for request notifications through the settings module. The user can switch request notification updates on or off when a request has been updated and if the notification is delivered via push notification, email or text. The user can also change the frequency of the request notification to be delivered immediately, hourly or daily at a specified time.

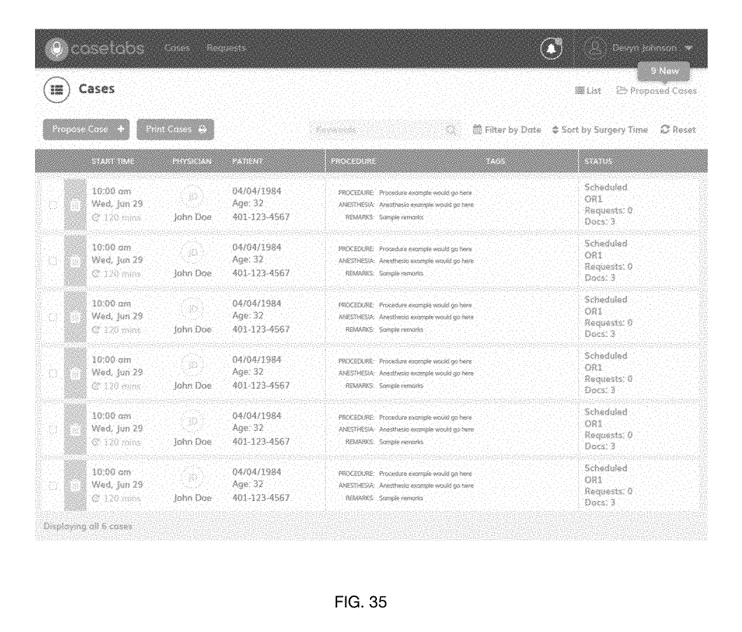

FIG. 35 depicts an example of an embodiment according to the present invention, which describes the propose a case module. In accordance with the preferred embodiment of the present invention, the user has the option to propose a case through the case list data overview. The user can also view and review new proposed cases by selecting the proposed cases data file within the case list data overview.

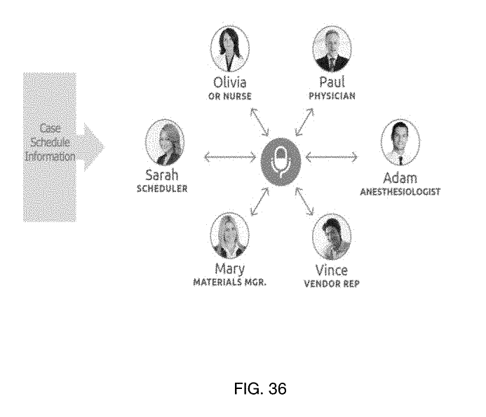

FIG. 36 depicts an example of an embodiment according to the present invention, which describes the various user groups of the application. In accordance with the preferred embodiment of the present invention, the client can assign various users related to cases to access and interact within the application interface. Different users who are granted access include but are not limited to: the Physician; the OR Nurse; the Anesthesiologist; The Vendor Representative; the Materials Manager; and the Scheduler.

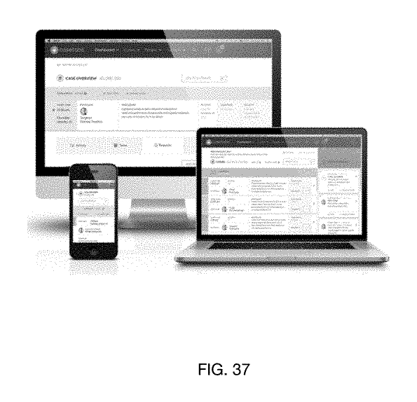

FIG. 37 depicts an example of an embodiment according to the present invention, which describes the various platforms available to the user to access the application. In accordance with the preferred embodiment of the present invention, the user can access the application interface across a variety of platforms, that include but are not limited to a desk top workstation, laptop computer and mobile device.

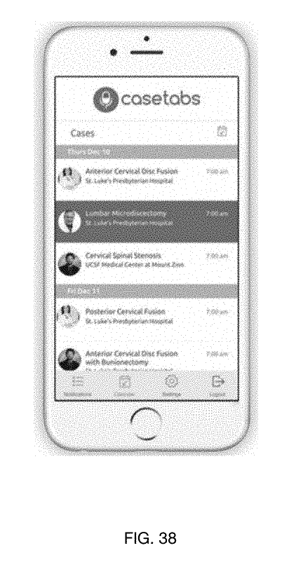

FIG. 38 depicts an example of an embodiment according to the present invention, which describes the use of the application on a mobile device. In accordance with the preferred embodiment of the present invention, the user can access the application interface through his or her mobile device.

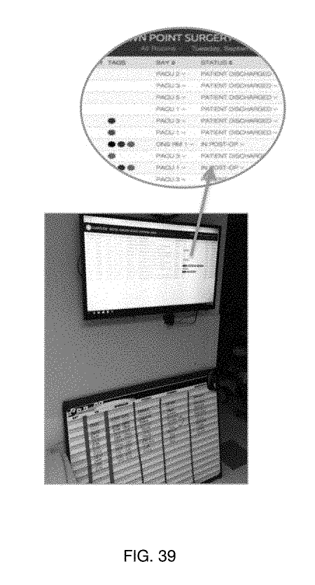

FIG. 39 depicts an example of an embodiment according to the present invention, which describes the digital surgery board feature of the present invention. In accordance with the preferred embodiment of the present invention, digital surgery boards are utilized by the client as a means of digital display of real time, updated case list data collected from the application interface.

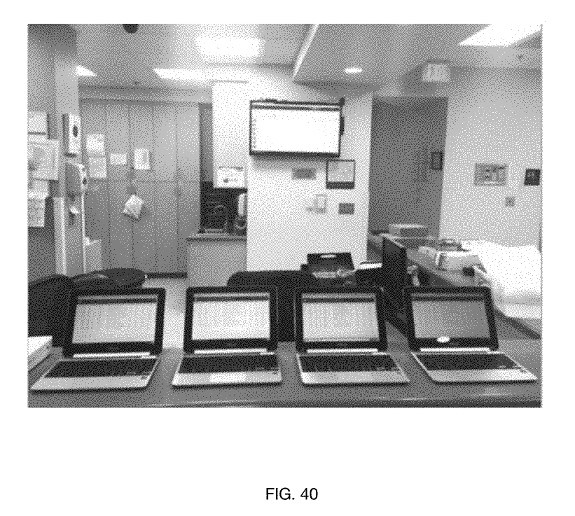

FIG. 40 depicts an example of an embodiment according to the present invention, which describes the digital surgery board feature of the present invention. In accordance with the preferred embodiment of the present invention, the client can utilize digital display boards to display updated case list data from the application interface in real time.

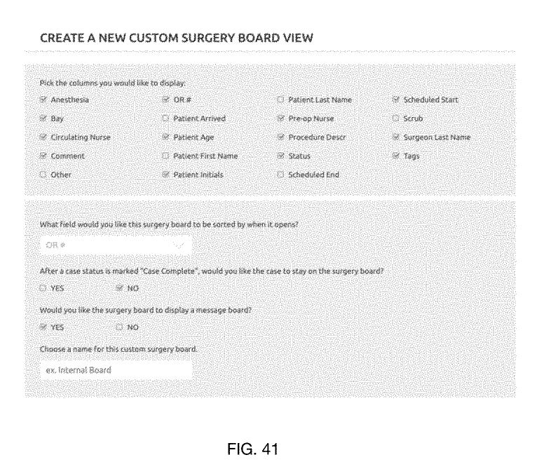

FIG. 41 depicts an example of an embodiment according to the present invention, which describes the digital surgery board feature of the present invention. In accordance with the preferred embodiment of the present invention, the client administrator can access the digital surgery display board settings module to select the data to be displayed and updated on a specific digital display board.

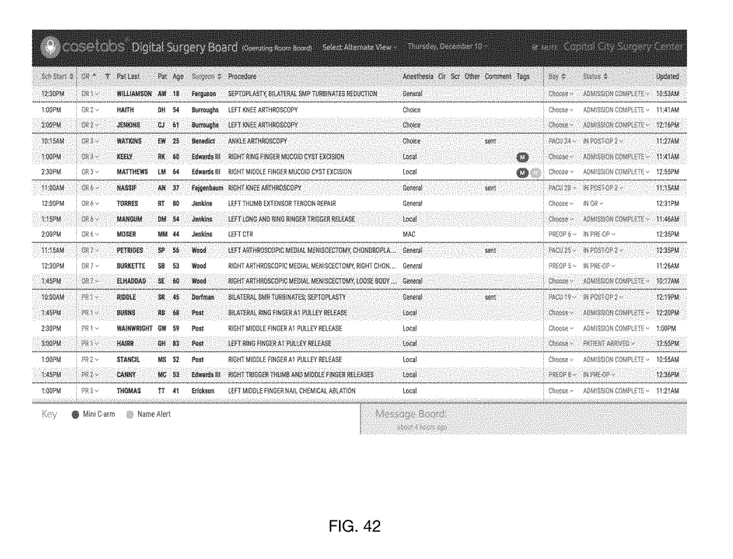

FIG. 42 depicts an example of an embodiment according to the present invention, which describes the digital surgery board feature of the present invention. In accordance with the preferred embodiment of the present invention, the digital display board can show an overview of upcoming and in progress cases as they are updated in real time.

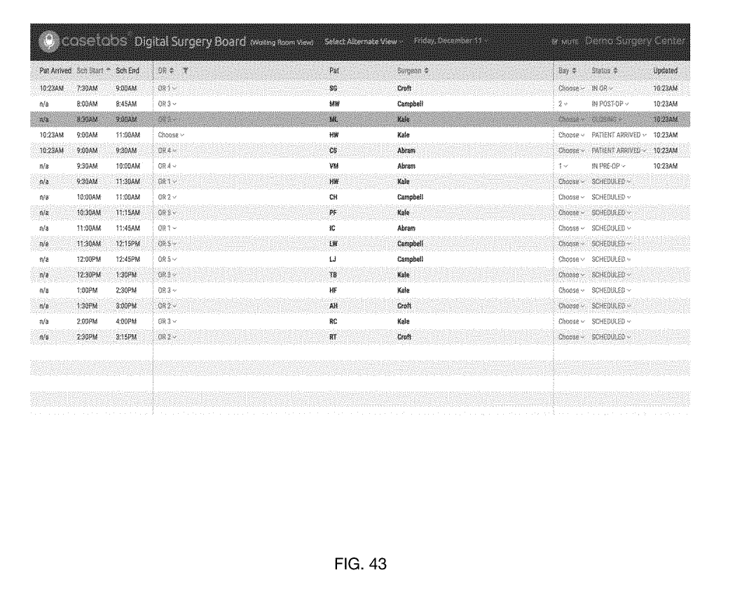

FIG. 43 depicts an example of an embodiment according to the present invention, which describes the digital surgery board feature of the present invention. In accordance with the preferred embodiment of the present invention, the digital display board can be configured to show cases in progress, in chronological order, as well as the individual assigned to the case and the location of the case.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that may be included in the disclosed technology. The disclosed technology is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the technology disclosed herein. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that may be included in the disclosed technology. The disclosed technology is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the technology disclosed herein. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, may be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration.

What is claimed is:

1. A computer-implemented method of displaying real-time scheduling and resource allocation information in connection with performing medical procedures, the method comprising:
receiving patient data indicative of patient identity and patient medical service and treatment requirements, the patient data having associated biometric data and associated electronic medical records and an intended course of human intervention and related patient medical treatment supplies; offering said patient a choice of time slots for having said medical procedure performed in connection with assessing said data indicative of intended course of human intervention and related patient medical treatment by evaluating the availability of specific human intervention participants and be evaluating the availability of related patient medical treatment supplies;
establishing a schedule for performing said medical procedure for the benefit of said patient, based on an optimal availability of all resources including a location for performing said medical service, locating and reserving said related patient medical treatment supplies and securing the availability of various medical professionals for performing said intended course of human intervention for the benefit of said patient;
reporting from a time of scheduling a medical procedure through performance of said medical procedure and through the recovery period of said patient after performance of said procedure, wherein said reporting is carried out by electronic display boards located proximate to a medical procedure site and via handheld electronic display devices in the possession of related medical procedure staff; and
displaying all of said patient data and scheduling data via handheld electronic displays and via fixed electronic displays associated with physical locations where medical procedures are performed.

2. The method according to claim 1 wherein the step of establishing a schedule includes taking into account the availability of certain medical professional staff members associated with a particular group of medical professionals.

3. The method according to claim 1 wherein the step of establishing a schedule includes taking into account the availability of said patient related medical treatment supplies.

4. The method according to claim 3 wherein said patient related medical treatment supplies include pharmaceutical products. The method according to claim 1 wherein medical professionals possessing smartphone devices may be queried as to theft availability to perform various medical procedures, offered the opportunity to undertake responsibility for participating in various medical procedures and committing to be engaged in connection with said various medical procedures and wherein a team of medical professionals required to perform said medical procedures for the benefit of said patient.

6. The method according to claim 6 wherein a medical procedure administrator regulates which medical professionals are selected for performing a particular medical procedure, and wherein said administrator regulates a manner in which a patient is informed of the results of said selection process.

7. The method according to claim 6 wherein said medical professionals are notified of said schedule via a srnartphone and wherein said medical professionals are provided with an ability to notify said administrator that availability criteria have changed in order that said administrator may select an alternative medical professional to perform said medical procedure.

8. A computer-controlled system for operating a real-time scheduling display interface and resource allocation platform interface and in connection with performing medical procedures, the system comprising:
a plurality of input devices for receiving patient data indicative of patient identity and patient medical service and treatment requirements, the patient data having associated biometric data and associated electronic medical records and an intended course of human intervention and related patient medical treatment supplies;
offering said patient a choice of time slots for having said medical procedure performed in connection with assessing said data indicative of intended course of human intervention and related patient medical treatment by evaluating the availability of specific human intervention participants and be evaluating the availability of related patient medical treatment supplies;
a computer programmed to establish a schedule for performing said medical procedure for the benefit of said patient, based on an optimal availability of all resources including a location for performing said medical service, locating and reserving said related patient medical treatment supplies and securing the availability of various medical professionals for performing said intended course of human intervention for the benefit of said patient;
an electronic output driver for reporting from a time of scheduling a medical procedure through performance of said medical procedure and through the recovery period of said patient after performance of said procedure, wherein said reporting is carried out by electronic display boards located proximate to a medical procedure site and via handheld electronic display devices in the possession of related medical procedure staff; and
wherein said electronic output driver includes an interface for reporting said schedule data to handheld electronic displays used by said medical staff for performing said human intervention for the benefit of said patient; and
wherein electronic displays are used to inform patients or patient related constituencies within a surgery center as to that status of any surgical procedure,

9. The computer-controlled system according to claim 8 wherein the step of establishing a schedule includes taking into account the availability of certain medical professional staff members associated with a particular group of medical professionals.

10. The computer-controlled system according to claim 8 wherein the information technology cloud is utilized to permit entry of patient data, medical professional availability data and medical supply availability data from a plurality of locations and a plurality of input devices including smartphone input devices, and wherein electronic displays are fixed in place at a site where medical procedures are performed for a plurality of people to view a status of progress as to a medical procedure underway with respect to said patient.

11. The computer-controlled system according to claim 10 wherein data indicative of what surgical supplies will be required to complete a surgical procedure are compared with what surgical supplies are contained within the inventory associated with the site at which patient will undergo a surgical procedure, and wherein a surgical procedure administrator may control the allocation of surgical supplies to certain scheduled surgical procedures.

12. The computer-controlled system according to claim 8 wherein said medical procedure professionals are assigned to a particular medical procedure associated with one of said patients by an administrator and said medical professionals are notified of said assignments via smartphones, and wherein said medical professionals may enter via said smartphones their status and availability for said scheduled procedures so that schedules may be altered in the case a selected medical professional is unavailable and said administrator may locate a replacement medical professional to perform said scheduled medical procedure.

13. A computer-controlled system for operating a real-time scheduling and resource allocation platform in connection with performing medical procedures and an interface for displaying real time information pertaining to the status of a surgical procedure to be used by patients or patient constituencies and used by medical professionals, the system comprising:
a plurality of input devices for receiving patient data indicative of patient identity and patient medical service and treatment requirements, the patient data having associated biometric data and associated electronic medical records and an intended course of human intervention and related patient medical treatment supplies;
offering said patient a choice of time slots for having said medical procedure performed in connection with assessing said data indicative of intended course of human intervention and related patient medical treatment by evaluating the availability of specific human intervention participants and be evaluating the availability of related patient medical treatment supplies;
a computer programmed to establish a schedule for performing said medical procedure for the benefit of said patient, based on an optimal availability of all resources including a location for performing said medical service, locating and reserving said related patient medical treatment supplies and securing the availability of various medical professionals for performing said intended course of human intervention for the benefit of said patient;
an electronic output driver for reporting from a time of scheduling a medical procedure through performance of said medical procedure and through the recovery period of said patient after performance of said procedure, wherein said reporting is carried out by electronic display boards located proximate to a medical procedure site and via handheld electronic display devices in the possession of related medical procedure staff, and wherein said electronic output driver includes an interface for reporting said schedule data to handheld electronic displays used by said medical staff for performing said human intervention for the benefit of said patient; and
wherein a plurality of said input and said electronic output drivers reside in handheld smartphones or electronic tablets and wherein a plurality of said electronic out drivers are deployed in association with large fixed digital displays or monitors disposed within or proximate to a surgical center so that medical staff, patients and other interested constituencies may freely see the status of each of said patients within a surgery center.

14. The computer-controlled system according to claim 13 wherein the step of establishing a schedule includes taking into account the availability of certain medical professional staff members associated with a particular group of medical professionals.

15. The computer-controlled system according to claim 13 wherein the information technology cloud is utilized to permit entry of patient data, medical professional availability data and medical supply availability data from a plurality of locations and a plurality of input devices including smartphone input devices, and wherein electronic displays are fixed in place at a site where medical procedures are performed for a plurality of people to view a status of progress as to a medical procedure underway with respect to said patient.

16. The computer-controlled system according to claim 15 wherein data indicative of what surgical supplies will be required to complete a surgical procedure are compared with what surgical supplies are contained within the inventory associated with the site at which patient will undergo a surgical procedure, and wherein a surgical procedure administrator may control the allocation of surgical supplies to certain scheduled surgical procedures.

17. The computer-controlled system according to claim 13 wherein said medical procedure professionals are assigned to a particular medical procedure associated with one of said patients by an administrator and said medical professionals are notified of said assignments via smartphones, and wherein said medical professionals may enter via said smartphones their status and availability for said scheduled procedures so that schedules may be altered in the case a selected medical professional is unavailable and said administrator may locate a replacement medical professional to perform said scheduled medical procedure.

18. The computer-controlled system according to claim 17 wherein video compression is used to enable distribution of medical procedure information across a plurality of user interfaces, optimized based on the video display interfaces selected for use by any of a set of users of the present invention.

19. The computer-controlled system according to claim 17 wherein artificial intelligence is used to sort data associated with said scheduled procedures and then to provide information indicative of a potential human error, and wherein said artificial intelligence includes a correction process in order to correct said human error.

20. The computer-controlled system according to claim 17 wherein RF beacons are deployed throughout a surgical facility to enable fixed patient status displays to synchronize with the handheld device displays used according to the present invention.
